(19) United States
(12) Patent Application Publication
(10) Pub. No.: US 2023/0248652 A1
(43) Pub. Date: Aug. 10, 2023

(54) BIODEGRADEABLE MULTI-CAVITY MICROPARTICLES AND THEIR USE IN TREATMENT

(71) Applicant: OXFORD UNIVERSITY INNOVATION LIMITED, Oxfird (GB)

(72) Inventors: James KWAN, Oxford (Oxfordshire) (GB); Umesh Sai JONNALAGADDA, Singapore (SG); Xiaoqian SU, Singapore (SG); Ipshita GUPTA, Singapore (SG)

(21) Appl. No.: 18/010,219

(22) PCT Filed: Jun. 16, 2021
(86) PCT No.: PCT/GB2021/051505

§ 371 Date: Dec. 14, 2022

(30) Foreign Application Priority Data
Jun. 16, 2020 (SG) 10202005705P
Mar. 23, 2021 (SG) 10202102971R
May 14, 2021 (GB) 2106915.8

(51) Int. Cl. A61K 9/16; A61K 31/573; A61K 31/436; A61K 9/00; A61L 29/16; A61P 9/10

(52) U.S. Cl. CPC A61K 9/1647; A61K 9/0009; A61K 31/436; A61K 31/573; A61L 29/16; A61P 9/10

(57) ABSTRACT

The present invention provides a core-shell microparticle comprising a biodegradable polymer with at least two or more surface cavities for use in the treatment of vascular disease, wherein the shell further comprises one or more drugs. The present invention further provides a core-shell microparticle which can be used for such treatments, the microparticle comprising a biodegradable polymer with at least two or more surface cavities, wherein the shell further comprises one or more drugs, wherein the one or more drugs are selected from anti-inflammatory drugs, immunosuppressants, anti-proliferative drugs, anti-coagulants and combinations thereof.

### FIELD OF INVENTION

The present invention relates to a core-shell, multi-cavity, biodegradable microparticle for use in the treatment of site-specific diseases. The particles are especially useful in the treatment of vascular diseases. The present invention also provides a drug-loaded, core-shell, multi-cavity, biodegradable microparticle.

### BACKGROUND

Vascular diseases account for some of the highest mortality rates worldwide, and incidences have been dramatically increasing due to lifestyle-related factors and aging populations. Some vascular diseases are caused by accumulation of lipids and fibrous tissues known as atherosclerosis. The accumulation of lipids can lead to the build-up of atherosclerotic plaques within the arterial intima, restricting blood flow. These plaques are formed from the build-up of foam cells that develop from monocytes attracted to the lipid rich subendothelial part of the arteries. The monocytes then differentiate into macrophages and excessively uptake and metabolise modified lipoproteins, and in turn further differentiate into foam cells. These foam cells accumulate over time and combine into a plaque that slowly becomes at risk for ruptures that occlude blood vessels and cause serious health implications. One example of a vascular disease, which may be caused by atherosclerosis, is peripheral artery disease (PAD).

In some cases vascular diseases associated with atherosclerosis can be treated by invasive surgical techniques such as insertion of stents or angioplasty. Trauma caused by these techniques can lead to “healing” tissue growth in the treated arteries, which causes them to narrow, restricting blood flow. This is known as restenosis.

Vascular diseases associated with atherosclerosis are typically site-specific and chronic diseases. For these site-specific diseases, it is desirable to non-invasively and locally deliver therapeutics over extended periods of time. This had led to a shift towards targeted drug delivery with sustained-delivery systems, which can maximise therapeutic impacts while keeping side effects to a minimum.

Drug-loaded vesicles have potential in achieving sustained and localised drug delivery. Such vesicles include liposomes, polymer micro/nanospheres, and ultrasound-sensitive micro/nanostructures. The small size, large surface area to volume ratio, and mobility in tissue of drug-loaded vesicles make them an ideal choice for targeted therapy by aiding in (i) penetration across blood tissue barriers, (ii) organ biodistribution, and (iii) cellular uptake. However, current drug delivery methods are limited due to low efficiency in targeting, image-guided positioning accuracy, and distribution of therapeutics through lesion sites.

High intensity focused ultrasound (HIFU) is a minimally invasive therapeutic technique which can be used to improve drug distribution and localised release. Ultrasound can be used to mediate drug delivery, often via acoustic cavitation, i.e., the dynamic oscillations of gas or vapour bubbles. Yet, to nucleate cavitation requires substantial acoustic energy capable of damaging off-target healthy tissue. Therefore preformed cavitation nuclei such as small gaseous particles (microbubbles) are widely used to enable cavitation at reduced acoustic pressure amplitudes. As such microbubbles have been shown to nucleate cavitation and improve the local delivery of therapeutic agents, including those for use in treatment of vascular disease.

However, despite the many benefits they provide, microbubbles have disease specific limitations owing to their large size and rapid destruction in an acoustic field. This rapid destruction means that treatment and drug delivery does not continue after HIFU has stopped. This means that microbubbles are not well suited for chronic conditions which need regular treatment over extended periods of time.

Therefore, to treat chronic and site-specific vascular diseases, there remains a need for targeted treatments that provide sustained drug delivery for a period of time after administration.

### SUMMARY OF THE INVENTION

The present inventors have developed a core-shell microparticle comprising a biodegradable polymer with at least two or more (i.e. at least two) surface cavities for use in treatment of vascular disease, in particular for use in the treatment of arterial inflammation, wherein the shell further comprises one or more drugs. The particle can be localised at a diseased site, using pressure waves such as HIFU, and degrades slowly to release the loaded drug. Thus the invention provides a means for both site-specific and prolonged treatment without multiple administrations, for use in improved treatment of vascular diseases.

Surprisingly, it has been found that the herein-described drug-loaded core-shell microparticle can reduce the presence of inflammatory cytokines, and volume of atherosclerotic plaques, in in vitro foam cell spheroid models. The effect of this treatment has been shown to be significantly improved compared to the use of drug alone. It has never previously been demonstrated that a drug-loaded microparticle can successfully reduce inflammation in this way. Thus, the invention also provides a core-shell microparticle comprising a biodegradable polymer with at least two or more (i.e. at least two) surface cavities for use in treating (i.e. reducing) arterial inflammation, wherein the shell further comprises one or more drugs. The treatment is typically useful in a subject suffering from vascular disease, in particular in a subject suffering from atherosclerosis or a subject who has received angioplasty treatment, e.g. a subject in need of treatment to prevent restenosis.

The biodegradable polymer of the particle can be tuned such that the particle degrades slowly. The inventors have therefore shown that it is possible to tailor the microparticle of the invention to release drugs over an extended period of time, at a rate selected for the treatment of a chronic condition, such as chronic vascular diseases.

Currently, cavitation nuclei being explored for their ability to deliver a therapeutic are limited to either gaseous microparticles, phase change droplets, or non-degradable solid nuclei. Our technology provides an improvement on all of these particles. With regard to microbubbles and phase change droplets, our particle is not destroyed during ultrasound exposure and can sustain cavitation for nearly 10 minutes. Furthermore, our particle is capable of being embedded into tissue for direct delivery of the therapeutic at the site of injury. With regard to solid cavitation nuclei (such as nanocups or mesoporous silica), the proposed particle is composed of a biodegradable material. This enables its contents to be released over long periods of time. Therefore, this particle will have an advantage in delivering therapeutic agents to sites of vascular disease, whereby drug distribution is a challenge, without having to be concerned with the side effects of a non-degradable particle being situated at the site of injury.

The present invention also provides a core-shell microparticle comprising a biodegradable polymer with at least two or more surface cavities, wherein the shell further comprises one or more drugs, wherein the one or more drugs are selected from anti-inflammatory drugs, immunosuppressants, anti-proliferative drugs, anti-coagulants and combinations thereof.

In some embodiments the invention provides a pharmaceutical composition comprising a plurality of microparticles as described herein and a pharmaceutically acceptable carrier or diluent. Also provided is a pharmaceutical composition for use in the treatment of vascular disease, in particular for use in the treatment of arterial inflammation, wherein the pharmaceutical composition comprises a plurality of microparticles as described herein, and a pharmaceutically acceptable carrier or diluent.

The invention also provides:
1. A biodegradable micro- or submicron-sized particle with at least two or more surface cavities present.
2. The surface cavities are capable of trapping gas and can reduce the threshold to nucleate cavitation.
3. The resulting particles are capable of being embedded into tissue upon exposure to ultrasound at frequencies above 100 kHz.

### BRIEF DESCRIPTION OF FIGURES

FIG. 1 shows SEM images of PLGA microparticles under different concentrations of PBS and PVA. The scale bar in the top left square represents 1 µm.

FIG. 2 shows SEM images of PLGA microparticles prepared using the double emulsion-diffusion-evaporation method exhibiting a multitude of diameters and porosity proportional to the concentration of porosigen (PBS) and stabilizer (PVA), that have been labelled into 4 categories, based on their morphology and diameter namely a) non-porous hollow spheres, b) porous hollow spheres, c) large multi-cavity particles (> 5 µm in diameter), and d) small multi-cavity particles (<5 µm in diameter).

FIG. 3 shows a schematic of a HIFU setup as used in Example 2.

FIG. 4 shows pictures of (A) the agarose flow chamber and (B) the porcine artery sample chamber as used in Example 2.

FIG. 5 shows the harmonic (upper line) and broadband (lower line) cavitation intensity of PLGA microparticles exposed to 1.1 MHz HIFU.

FIG. 6 compares the effect of concentrations of PBS and PVA in the particle formulations on the rates of RhB release. The release of RhB for all formulations tested followed a rapid rate of release within the first 24 hours, after which there was a stagnation of release.

FIG. 7 shows SEM images mcPLGA MPs degrading across 15 days.

FIG. 8 shows penetration tests of “drug” loaded mcPLGA MPs. Once embedded into the agarose, the particles release the “drug”, as indicated by a decay in fluorescent intensity over 15 days.

FIG. 9 shows quantification of fluorescent intensity of FIG. 8 at both 37 C and 4 C.

FIG. 10 shows RhB-mcPLGA MPs penetration in porcine artery. (A) Control artery with no RhB-mcPLGA MPs flow and ultrasound exposure (B) Control artery with only RhB-mcPLGA MPs flow and no ultrasound exposure (C) Artery with RhB-mcPLGA MPs flow and ultrasound exposure shows penetration of RhB-mcPLGA MPs into the inner arterial wall. The dotted line boxes of A, B and C shows zoomed in images. (D) Confocal laser scanning microscope Z-stack imaging of test artery 50 µm section showing location of RhB-mcPLGA MPs. These fluorescent images confirm that particles are embedded into the tissue and are not artifacts from sampling procedure. The dotted lines of D outline the endothelial and sub-endothelial region where most of RhB-mcPLGA MPs were located. RhB fluorescence is observed within the dotted lines of D.

FIG. 11 shows histopathological analysis by H&E staining of porcine arteries under (A) no RhB-mcPLGA MPs or ultrasound exposure, (B) RhB-mcPLGA MPs passed through the artery without ultrasound exposure, (C) no RhB-mcPLGA MPs with ultrasound exposure to the artery, and (D) RhB-mcPLGA MPs passed through the artery with ultrasound exposure. The fluorescence and H&E staining images of artery section with RhB-mcPLGA MPs passed through the artery with ultrasound exposure showed no signs of damage to the endothelium of the porcine artery indicating the safety of this technique.

FIG. 12 shows fluorescence images of porcine arteries under (Top panel) RhB-mcPLGA MPs passed through the artery without ultrasound exposure, and (Bottom panel) RhB-mcPLGA MPs passed through the artery with ultrasound exposure. The fluorescence images of artery section with RhB-mcPLGA MPs passed through the artery with ultrasound exposure show no signs of damage to the endothelium of the porcine artery, indicating the safety of this technique.

FIG. 13 shows a fluorescent image of a 3D foam cell spheroid exposed to DAPI-RhB-mcPLGA MPs and ultrasound one day after remote implantation.

FIG. 14 shows oil Red O staining of cytoplasmic lipid droplets showing the effect of Dex on lipid accumulation in foam cell spheroids.

FIG. 15 shows a schematic representation of the therapeutic ultrasound experimental set-up as used in Example 3.

FIG. 16 shows representative images of the normalized spectral density curves for three different shapes of particles, namely the a) spherical and both the b) small and c) large multicavity particles. As is seen, the multicavity particles (see b & c) transitioned from stable to inertial cavitation at much lower pressures in comparison to the nonporous spherical variants.

FIG. 17 shows the intensity of harmonic (upper line) and broadband (lower line) emissions observed for all microparticle formulations, and their dependence on both the diameter of the microparticle and acoustic intensity.

FIG. 18 shows the estimated dependence of acoustic pressure amplitude required to achieve 50% probability of (a) harmonic and (b) broadband cavitation on the diameter of particles. The dependence on particle diameter can be observed indicating the effect of Laplace pressure on cavitation threshold most strikingly for the porous hollow spheres. tested.

FIG. 19 shows the probability of cavitation for all microparticle formulations, and the dependence of probability of cavitation on both the diameter of the microparticle and acoustic intensity. Left hand lines indicate stable cavitation thresholds, right hand lines indicate inertial cavitation thresholds.

FIG. 20 shows a) a schematic representation of the diagnostic ultrasound experimental setup. b) a schematic for selection of region of interest (ROI) for vessel and tissue. CTR analysis was done by calculating the average of pixel intensity in the four tissue and two vessel ROIs selected. This was done to minimize variability.

FIG. 21 shows samples as imaged with the diagnostic ultrasound scanner and the corresponding contrast to tissue ratio (CTR) values in dB with reference to deionized water. As is seen, highest enhancement (> 20 dB) was observed for the larger multicavity particles (diameter > 5 µm) i.e., category (c): the right top corner and in the sample in the middle of the matrix.

FIG. 22 shows the measured CTR for representative microparticles from the different morphology groups (2 µm in diameter smooth spheres (labelled non-porous spheres), 2 µm in diameter multi-cavity microparticles (labelled smaller multicavity particles), and 6 µm in diameter multi-cavity microparticles (labelled larger multicavity particles)) in addition to deionized water for increasing input pressures from 10% to 100% power (corresponding MI values of 0.11 to 1.1).

FIG. 23 shows the representative images and CTR analysis for Example 5, at varying concentrations and volumes of stabiliser (PVA) and porosigen (PBS).

FIG. 24 shows representative (a) SEM, (b) TEM, and (c) fluorescence images of Dex-loaded mcPLGA MPs (Dex/mcPLGA MPs). For (c) rhodamine B (RhB) was also co-loaded to track the location of particles. (d) shows the size of mcPLGA MPs as measured by Dynamic Light Scattering (DLS). (e) shows the release of Dex from mcPLGA MPs in PBS at 37° C. Scale bars represent 1 µm. 3 independent experimental sets were performed.

FIG. 25 shows the acoustic response of mcPLGA MPs. (a) shows representative spectral density curves of mcPLGA MPs under different exposure pressures. (b) shows cavitation intensity (upper line harmonic, lower line broadband) and (c) show probability of cavitation of mcPLGA MPs exposed to HIFU at 1.1 MHz (left line harmonic, right line broadband).

FIG. 26 shows contrast enhancement from mcPLGA MPs. (a) B mode ultrasound imaging with the diagnostic ultrasound scanner of DI water and 1 mg/ml suspensions of hollow sphere PLGA (hsPLGA) microparticles and mcPLGA MPs. (b) Corresponding CTR values of DI water, hsPLGA, and mcPLGA MPs (mean ± SD, n = 3). ** denotes a p < 0.01 and *** denotes a p < 0.001.

FIG. 27 shows (a) spheroids (indicated by the smaller cycles in the top left square) embedded in alginate beads (indicated by the dashed cycles in the top row). (b) B-mode ultrasound images of spheroids embedment before and after HIFU treatment. ROI are marked on the top row by dashed cycles. (c) Confocal images of extracted foam cell spheroids. (Left) An image of a foam cell spheroid treated with 1 mg/ml mcPLGA MPs without HIFU exposure. (Middle) A similar foam cell spheroid treated with 1 mg/ml mcPLGA MPs without HIFU exposure but stained with DAPI to emphasize the diffusion limitation of cell spheroids. (Right) An image of a foam cell spheroid treated with 1 mg/ml mcPLGA MPs (loaded with DAPI and RhB) after HIFU exposure (right). The scale bars represent 100 µm. In the bottom row, the lighter grey around the edge of the cycle is DAPI, the darker spots within the cycle are RhB, demonstrating penetration and distribution throughout the spheroid of MPs post HIFU.

FIG. 28 shows Oil Red O and haematoxylin staining of foam cell spheroids in different conditions. The scale bars represent 50 µm. The figure shows how oil droplets near the core of the foam cell spheroid were still present after treatment with Dex, due to diffusion limitations (right centre image). The right hand image shows HIFU propelled Dex/mcPLGA MPs produced a more dramatic and uniform reduction of oil droplets compared to all the other treatment groups.

FIG. 29 shows evaluation of cytokine release from foam cell spheroids. THP-1 derived foam cell spheroids exposed to HIFU+mcPLGA MPs, Dex alone, HIFU+Dex/mcPLGA MPs or untreated. Graphs showing quantification of the cytokine secretions categorized as either early-stage inflammation, or late-stage inflammation. * denotes a p < 0.05 between the indicated groups and untreated controls. Data presented are representative of 3 independent experiments.

### DETAILED DESCRIPTION OF THE INVENTION

### Microparticles

The core-shell microparticle is a multi-cavity particle comprising a biodegradable shell surrounding a core. In some preferred embodiments the core is a hollow core. In some embodiments the core-shell microparticle is not spherical. In one embodiment, each biodegradable microparticle may comprise between 2 to 5 surface cavities. The multi-cavity particles may have cavities in a variety of different forms. For instance, the cavities may be in the form of cups, pores, or tunnels which go through the particles. Further, the surface cavities of the invention may be indentations on the shell, and/or they may form a hierarchical porous shell with the hollow core, wherein the resultant hierarchical porous shell may be cage-like and/or they may form tunnels to the core.

Thus, surface cavities may:
i) be indentations on the shell; and/or
ii) form tunnels to the core; and/or
iii) result in a hierarchical porous cage-like shell around the core.

In one embodiment, a biodegradable microparticle comprises a) a plurality of surface cavities; and b) a gas pocket present in some or all of the surface cavities. The multi-cavity structure may contain from 2 to 20 cavities, preferably from 2 to 10 cavities, more preferably from 2 to 5 cavities.

The core-shell microparticle typically has an average diameter of 10 µm or less, preferably 6 µm or less, more preferably 5 µm or less. Typically, a microparticle has a diameter of at least 0.1 µm, preferably at least 0.5 µm, more preferably at least 1 µm. A suitable microparticle therefore has a diameter from 0.1 to 10 µm. In preferred embodiments, the core-shell microparticle has an average diameter of from 1 µm to 6 µm.

Microparticles above this size give rise to a risk of blockages in peripheral arteries, due to the particle size approaching the same diameter as these arteries. Microparticles below this size, in contrast, may be unable to carry an appropriate amount of drug, meaning that more particles need to be administered to provide an effective dosage for treatment. Smaller particles are also more difficult to visualise using ultrasound imaging, post-administration. Larger particles therefore have the advantage that ultrasound imaging techniques can more effectively be used to determine if microparticles are embedded in the desired site.

Typically the cavities of the multi-cavity microparticles have an average diameter of from 0.1 to 1.0 µm, for example from 0.4 µm to 0.8 µm, preferably from 0.65 µm to 0.75 µm. Diameter of cavities may be measured by, for instance, SEM.

The shell of the core-shell microparticle typically comprises a biodegradable polymer. As used herein, a biodegradable polymer is typically a polymer which degrades over time in a biological medium, for example blood, or tissue. The biodegradable polymer of the core-shell microparticle may be an aliphatic polyester, an aromatic copolyester, polyamide, poly(ester-amide), polyurethanes, polyanhydrides, polysaccharides, and blends or copolymers of the afore-mentioned examples.

When the biodegradable polymer is an aliphatic polyester, it is preferably poly(lactic-co-glycolic acid) (PLGA), polylactic acid (PLA), polyglycolic acid (PGA), polycaprolactone (PCL), poly(butylene succinate) and its copolymers, poly(p-dioxanone) (PPDO), poly(hydroxybutyrate) (PHB), polycarbonates, or blends or copolymers thereof. When the biodegradable polymer is an aromatic copolyester, it is preferably poly(butylene adipate-co-terephthalate) (PBAT) or a copolymer thereof. When the biodegradable polymer is a polysaccharide, it is preferably chitosan, cellulose, or hyalauronic acid, or blends or copolymers thereof. Preferably, the biodegradable polymer is an aliphatic polyester, or blend or copolymer thereof. More preferably, the biodegradable polymer is poly(lactic-co-glycolic acid) (PLGA), polycaprolactone or a blend or copolymer thereof. Even more preferably, the biodegradable polymer is PLGA.

PLGA polymers comprise a blend of lactic acid and glycolic acid. Depending on the relative ratios of these components, the rate at which the polymer degrades can vary. In the present invention, the ratio of lactic acid to glycolic acid can be varied in order to optimise degradation rate. For example, the ratio can be optimised such that the release of the one or more drugs occurs gradually over the course of around 28 days. This ability to tune the rate of degradation to provide sustained release of therapeutics, makes the present invention highly effective in the treatment of chronic conditions, such as peripheral artery disease, which would otherwise require regular and repeated administration - which is inefficient for healthcare providers and patients alike.

The PLGA polymer of the invention may comprise a blend of lactic acid to glycolic acid of from 1:99 to 99:1, preferably from 1:9 to 9:1, more preferably from 1:4 to 4:1, even more preferably from 2:3 to 3:2. In some embodiments of the invention, the biodegradable polymer consists of a lactic acid polymer. In some embodiments of the invention, the biodegradable polymer consists of a glycolic acid polymer.

Lactic acid degrades more slowly than glycolic acid. Therefore, to achieve a longer period of degradation, the amount of lactic acid in a PLGA blend can be increased. This facilitates prolonged drug release, advantageous in the treatment of chronic disease. Conversely, if a shorter degradation period is desired, the amount of glycolic acid in a PLGA blend can be increased. Coatings may also be used to prevent degradation and provide slower release profiles.

The biodegradable nature of the materials used for the present invention is highly advantageous as it not only enables drug release, but prevents build-up of non-biodegradable polymers in the body, which can be toxic or harmful upon accumulation.

The shell of the core-shell microparticle further comprises one or more drugs. The shell of the core-shell microparticle may comprise one or more hydrophobic chemicals including drugs such as sirolimus, steroids, dexamethasone, etc. In some embodiments the one or more drugs are selected from anti-inflammatory drugs, immunosuppressants, anti-proliferative drugs, anti-coagulants and combinations thereof. In some embodiments the shell comprises a steroid, preferably dexamethasone. In some embodiments the shell comprises an immunosuppressant, for example sirolimus and/or everolimus, preferably sirolimus. In some embodiments, the shell comprises an anti-proliferative drug, typically a chemotherapy agent, for example a taxane, preferably paclitaxel. In some embodiments the anti-proliferative drug is sirolimus. In some embodiments the shell comprises an oligosaccharide, preferably hydroxyl beta cyclodextrin (HBCD). Combinations of two or more drugs may be present in the microparticles. In some embodiments the shell comprises a steroid. In some embodiments the shell comprises dexamethasone. In some embodiments the shell comprises sirolimus.

Typically the one or more drugs are drugs for use in the treatment of vascular disease, preferably for use in the treatment of atherosclerosis and/or restenosis. Preferably, the shell comprises one or more anti-inflammatory drugs. In some embodiments at least one anti-inflammatory drug is a steroid, preferably a glucocorticoid, more preferably dexamethasone.

### Synthesis

Typically, the microparticles are prepared using a double emulsion procedure. The first step of the synthesis of the microparticles typically comprises dissolving the at least one biodegradable polymer, and the at least one drug (one or more drugs) as discussed above, in an appropriate solvent to produce an organic phase. Construction of the biodegradable polymer is such that it allows its payload to be delivered at the site of the disease and not within the blood vessel network. In an exemplary embodiment this first step comprises dissolving PLGA in dichloromethane (DCM).

Typically this step is followed by addition of an aqueous phase to the organic phase and mixing (for example by sonication) to form a water-in-oil (W/O) emulsion. The aqueous phase may comprise a porosigen, for example phosphate buffered saline (PBS).

The next step to obtaining the microparticles of the invention is typically to combine the W/O emulsion with a further aqueous phase and homogenise the resulting mixture to produce a W/O/W emulsion. The further aqueous phase typically comprises a stabiliser such as poly(vinyl alcohol) (PVA). The organic solvent may then be allowed to evaporate from the W/O/W emulsion.

In order to trap gas, the particles of the invention are typically dried in air (or an alternative gas) to form a gas bubble. The particles may then be re-suspended. The drying and re-suspending process allows the particles of the invention to cavitate.

The microparticles of the invention can then be collected, for example by centrifugation, and optionally washed, for example with distilled water, and redispersed.

The microparticles of the invention may then be lyophilised to achieve a dried powder for long term storage.

Typically, gas bubbles form when dried or lyophilised microparticles are suspended in a liquid. Thus the invention provides a core-shell micro-particle comprising a biodegradable polymer with at least two or more surface cavities in which gas bubbles form upon mixing the core-shell microparticle in a liquid. Further, the invention provides a core-shell microparticle according to the previous statement, wherein the gas bubbles nucleate cavitation. Therefore, the invention provides multi-cavity microparticles suspended in a liquid medium whereby at least one, preferably at least two, cavities contain a gas bubble.

The encapsulation efficiency of the microparticle is a measure of the amount of drug which can be absorbed into the microparticle shell. The encapsulation efficiency is determined as the amount of drug which is incorporated into the microparticle, given as a percentage of the total amount of drug dissolved in the organic phase prior to preparation of the microparticle. Typically, the encapsulation efficiency of the microparticle can vary depending on the drug being loaded. Typically, the encapsulation efficiency ranges from about 30% to about 99%, for example from 40 to 90%, e.g. from 60 to 90% or 70 to 90%. For example, in the case of sirolimus the encapsulation efficiency is around 40 %, whereas in the case of dexamethasone the encapsulation efficiency is around 60 to 90 %, for example from 70 to 80%.

The drug loading efficiency of the microparticle is a measure of the amount of drug incorporated into the microparticle shell. The drug loading efficiency is determined as the mass of drug in the microparticle shell as a percentage of the total mass of the shell. Typically, the drug loading efficiency ranges from about 1 % to about 10 %, for example from 2 to 8 %, e.g. from 3 to 7 % or 4 to 6%.

### Tuneability

The core-shell microparticle has the advantage of tuneable size and morphology. This has allowed the present inventors to hone the parameters of the microparticles to optimise factors such as the cavitation threshold. This also allows for more flexible uses of the technology, which can be adapted for smaller particles (particle diameter < 5 µm) more suitable for therapeutic purposes, and larger particles (particle diameter > 5 µm) which are more suitable for diagnostics as they nucleate bubbles with a larger scattering cross section and behave as better contrast agents. Smaller variants are preferable for use in the therapeutic domain given their favourable size.

The shape and dimensions of the microparticle of the invention may be controlled by varying the concentration and composition of the various reagents used in synthesis of the particles.

The first aqueous phase typically comprises a porosigen. A porosigen may be any suitable material which will disperse or degrade to leave a porous network. For example, the porosigen may be a salt solution, such as a solution of a sodium and/or potassium salt, e.g. sodium chloride, potassium chloride, sodium phosphates such as sodium dihydrogen phosphate, potassium phosphates such as potassium dihydrogen phosphate, or mixtures thereof. PBS solution, which is a mixture of such salts, is a preferred porosigen. Suitable concentrations of porosigen in the first aqueous phase are from 0.01 to 0.5 M, e.g. 0.01 M to 0.2 M.

The shape of the microparticles of the invention can be controlled by adjusting the composition of the first aqueous phase, such as by adjusting the concentration of the porosigen. Generally the inventors have found that increasing the porosigen (typically the salt) concentration of the first aqueous phase produces microparticles of the invention with greater numbers and/or sizes of cavities. For example, using a first aqueous phase which is PBS having a 0.01 M salt concentration may produce microparticles with small, infrequent surface pores that do not penetrate the full depth of the shell, while using a first aqueous phase which is PBS have 0.1 M salt concentration may result in particles with higher numbers of cavities, as well as deeper cavities, possibly including pores and/or tunnels as well as surface cavities.

The size of the microparticles of the invention can also be controlled by adjusting the salt concentration of the porosigen of the aqueous component. Typically, the present inventors have found that an increased concentration of salt, such as PBS, in the porosigen results in a microparticles forming with a larger diameter. For example, using a first aqueous phase which is PBS having a 0.01 M salt concentration, and using 1 wt % PVA in the further aqueous phase, PLGA microparticles generally formed with an average diameter of roughly 2 µm. However, in an alternative exemplary synthesis it was found that with a first aqueous phase which is PBS have 0.1 M salt concentration, and 1 wt % PVA in the further aqueous phase, the PLGA microparticles generally formed with an average diameter of roughly 6 µm.

The further aqueous phase may comprise a stabilising agent. The stabilising agent may be any suitable material which can stabilise the water-oil interface. PVA is a preferred stabilising agent. A suitable amount of stabilising agent is from 1 to 10 wt% in the further aqueous phase.

The shape of the microparticle can be controlled by adjusting the concentration of the stabilising agent, such as by adjusting the weight percentage of PVA used. Conversely to the concentration of the porosigen, the present inventors found that increasing the weight percentage of stabilising agent used in the synthesis of the microparticles of the invention, reduced the depth of the surface cavities that formed.

The size of the microparticle of the invention can also be controlled by adjusting the concentration of the stabilising agent, such as by adjusting the weight percentage of PVA in the further aqueous solution. The present inventors have found that a higher weight percentage of stabiliser reduces the average diameter of microparticles synthesis. For example, in an exemplary embodiment of the invention, using a first aqueous phase which is PBS having a 0.2 M salt concentration, and using 1 wt % PVA in the further aqueous phase, PLGA microparticles generally formed with an average diameter of roughly 6 µm. In another exemplary embodiment of the invention, wherein the conditions and reagents were the same except that 10 wt% PVA was used, the PLGA microparticles were found to form with an average diameter of roughly 2 µm.

An ability to finely tune the size and morphology of the particles in the above ways is extremely advantageous, as it allows for microparticles of the invention with preferential properties for use in drug delivery.

### Use of the Particles of the Invention

Typically the microparticles and compositions described herein are for use in the treatment of vascular disease. Vascular disease, as used herein, is a disease affecting the arteries and/or veins of a subject. Typically the diseased site has an accumulation of lipids and fibrous tissues which restrict blood flow, wherein typically this build-up is in the form of foam cells.

In some embodiments the treatment of vascular disease comprises treatment of atherosclerosis, thrombolysis (blood clot destruction), anti-inflammatory drug delivery to damaged arteries post angioplasty, and/or prevention of restenosis through the delivery of anti-proliferation drugs (e.g. sirolimus). In some embodiments, the treatment comprises treatment of atherosclerosis, thrombolysis, treatment of damaged arteries post angioplasty (e.g. reduction in inflammation in damaged arteries) and/or prevention of restenosis. In some embodiments, the treatment comprises treatment of atherosclerosis, treatment of damaged arteries post angioplasty (e.g. reduction in inflammation in damaged arteries) and/or prevention of restenosis. Prevention of restenosis is typically through the delivery of anti-proliferation drugs, for example sirolimus. In preferred embodiments the vascular disease is peripheral artery disease (PAD), wherein typically treatment of PAD comprises treatment of atherosclerosis and/or prevention of restenosis. In some embodiments, treatment of PAD comprises treatment of atherosclerosis.

In some embodiments the microparticles and compositions of the invention are for use in the treatment of arterial inflammation. The treatment of arterial inflammation is typically in a subject suffering from vascular disease, in particular in a subject suffering from atherosclerosis or a subject who has received angioplasty treatment, e.g. a subject in need of treatment to prevent restenosis.

The present invention also provides a method for the treatment of vascular disease comprising administration to a subject in need of treatment, an effective amount of a core-shell microparticle comprising a biodegradable polymer with at least two or more surface cavities, wherein the shell further comprises one or more drugs. In some embodiments, the invention provides a method of treatment of atherosclerosis; a method of thrombolysis (blood clot destruction); a method of anti-inflammatory drug delivery to damaged arteries post angioplasty; and/or a method of prevention of restenosis through the delivery of anti-proliferation drugs (e.g. sirolimus). In some embodiments, the invention provides a method for the treatment of atherosclerosis and/or prevention of restenosis. In some embodiments, the invention provides a method of prevention of restenosis through delivery of anti-proliferation drugs, for example sirolimus. The present invention also provides a method for the treatment of (i.e. reduction of) arterial inflammation in a subject in need of treatment, comprising administering to said subject an effective amount of a core-shell microparticle comprising a biodegradable polymer with at least two or more surface cavities, wherein the shell further comprises one or more drugs.

The invention also provides use of a core-shell microparticle comprising a biodegradable polymer with at least two or more surface cavities, wherein the shell further comprises one or more drugs, in the manufacture of a medicament for use in the treatment or prevention of vascular disease. Typically the medicament is for use in treatment of atherosclerosis, thrombolysis (blood clot destruction), anti-inflammatory drug delivery to damages arteries post angioplasty, and/or prevention of restenosis through the delivery of anti-proliferation drugs (e.g. sirolimus). Typically the medicament is for use in the treatment of atherosclerosis and/or prevention of restenosis. Typically, the prevention of restenosis is through delivery of anti-proliferation drugs, for example sirolimus. The invention also provides use of a core-shell microparticle comprising a biodegradable polymer with at least two or more surface cavities, wherein the shell further comprises one or more drugs, in the manufacture of a medicament for use in the treatment of (reduction of) arterial inflammation.

Typically a microparticle or composition for use according to the invention is, (a) introduced into the vicinity of biological tissue; and (b) subjected to a pressure wave such that the core-shell microparticle is embedded into the biological tissue.

The biological tissue is generally a blood vessel, typically a diseased site within a blood vessel, wherein the diseased site is an area of a blood vessel wall which is affected by vascular disease as defined above. In particular the blood vessel wall may be affected by peripheral artery disease or atherosclerosis, or be susceptible to restenosis, or any combination thereof.

When embedded in the biological tissue, the microparticle slowly degrades to release its one or more drug at the diseased site. The microparticles of the present invention have been shown to greatly reduce the presence of inflammatory cytokines at the diseased site and to reduce the volume of foam cells present. This effect is not observed for administration of the drugs alone. Thus the microparticles are useful in the reduction of arterial inflammation, particularly the reduction of inflammatory cytokines. The reduction of inflammatory cytokines is significantly improved compared to the effect of administration of drugs alone. Thus the invention provides a surprisingly effective reduction in inflammation of diseased arteries, and thus is particularly effective in the treatment of diseases where such arterial inflammation is implicated, including atherosclerosis, peripheral artery disease, prevention of restenosis and other vascular diseases.

### Dosages of the Microparticle or Composition

The amount of drug, and therefore microparticle or composition, to be delivered can be determined by the skilled person by reference to known dosages for the relevant drug.

Microparticles and compositions comprising a therapeutically effective amount of a drug will be administered. A therapeutically effective amount of microparticle or composition will be administered. It will be understood that the specific dose level for any particular subject will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will usually be determined by clinical trial.

Typically, when the microparticle comprises dexamethasone, the microparticles or composition are administered to provide a drug dosage equivalent to from 0.5 mg to 50 mg daily, preferably from 0.5 mg to 15 mg daily, more preferably from 0.5 mg to 10 mg daily. Typically, when the microparticle comprises sirolimus, the microparticles or composition are administered to provide a drug dosage equivalent to from 1 mg to 6 mg daily, preferably from 2 mg to 4 mg daily.

### Administration of the Microparticle or Composition

There is no particular limit as to how the particles or compositions thereof may be introduced into a blood vessel provided it is via clinically safe means. For example, the particles may be administered in a variety of dosage forms, for example aqueous and oily suspensions. In some embodiments the core-shell microparticle of the invention is introduced into the vicinity of a biological tissue though intravenous injection, intramuscular injection, or catheter injection.

In some embodiments the microparticle or composition is introduced to a disease site within a blood vessel on the surface of a balloon, or via a catheter. In some embodiments the microparticle is introduced to a blood vessel on the surface of a medical balloon, such as concurrently with angioplasty. In some embodiments, the microparticle or composition is introduced via a catheter, such as by coating onto the surface of a catheter inserted into a vein or artery.

In some embodiments the microparticles or compositions of the invention are delivered via injection, for example by intravenous injection or via a catheter, typically directly into a blood vessel.

The particles are typically administered in the form of a pharmaceutically acceptable composition, together with a pharmaceutically acceptable carrier or diluent. In some embodiments, the carrier or diluent is water (sterile water) or an aqueous solution for example saline, phosphate-buffered saline (PBS), or a phosphate-buffered solution. Typically, the particles are provided in the form of a suspension, typically wherein the suspension is in water or aqueous solution, for example saline, or phosphate-buffered saline. When the microparticles are suspended in liquid, for example in a pharmaceutical composition, one or more gas bubbles may be present in the cavities of the microparticle, wherein the gas bubbles nucleate cavitation.

Advantageously, adjuvants such as a local anaesthetic, preservative and buffering agent can be dissolved in the carrier or diluent.

Further excipients, as deemed appropriate by the skilled person, could also be included. For example, optionally, the pharmaceutical composition further comprises an ultrasound contrast agent and/or a visual imaging agent.

In some embodiments the invention may be administered in combination with a separate ultrasound contrast agent and/or visual imaging agent. Typically an ultrasound contrast agent may be a microbubble, for example, a microbubble of perfluorocarbon, nitrogen gas, or sulfur hexafluoride stabilised in a phospholipid membrane. Typically a visual imaging agent may be a dye, for example, DAPI or Rhodamine-B (RhB). The microparticles may be administered separately from the ultrasound contrast agent and/or visual imaging agent, or they may be administered together with the ultrasound contrast agent/visual imaging agent in the same composition. Preferably, in embodiments wherein a separate ultrasound contrast agent and/or visual imaging agent is administered with the microparticles, they are administered in the same composition. A particular advantage of the invention, however, is that the microparticles themselves act as ultrasound contrast agents, and thus ultrasound imaging can be carried to determine the administration of particles at the desired site, without addition of further contrast agents.

### Exposure to Pressure Waves

Microparticles of the present invention are solid cavitation nuclei capable of being embedded into diseased tissues upon exposure to ultrasound.

Vascular diseases such as atherosclerosis, which contributes to peripheral artery disease, are often treated using drug coated balloons or drug coated stents. The balloons press the drug against the site where treatment is needed, but in conventional treatment the drug can be easily dispersed by the flow of the blood stream. The pressure wave delivered to the disease site in the present invention embeds the microparticles of the invention into the wall of the blood vessel, meaning the drug stays localised at the site where it is needed. This has been shown to greatly improve the effect in treating the disease compared to the administration of the drug alone. It also reduces the likelihood of any undesirable off-target side effects.

As the acoustic wave propagates through a liquid media, gas molecules may coalesce to form a bubble. This bubble can further interact with the acoustic field to oscillate in size, based the rarefaction and compression phases of the field. At low acoustic intensities, the bubble will oscillate symmetrically in a process called stable cavitation. Stable cavitation can perturb the surrounding fluid to create microstreams that facilitates drug transport.

Higher acoustic intensities will generate larger energy gradients, causing the bubble to grow. Eventually, the bubble will not retain its size and will collapse to produce inertial cavitation. The bubble collapse caused by inertial cavitation creates local physical and chemical changes. In the context of therapeutic ultrasound, these physical effects generated by inertial cavitation allow for local streaming effects to propel nanoparticles and enhance permeation of therapeutics to the desired tissue.

Such factors allow the microparticles (and thereby the payload of the biodegradable polymer) as used in the invention to be delivered at the site of the disease and not within the blood vessel network.

Thus, on application of ultrasound, the microparticle of the invention may undergo stable and/or inertial cavitation. The particles are thus capable of being embedded into the wall of a blood vessel, for example into a sub-endothelial region.

Typically, the pressure wave that the core-shell microparticle is subjected to is one or more selected from ultrasound, focused ultrasound, shockwaves, low intensity focused ultrasound, and high intensity focused ultrasound. Preferably the pressure wave that the core-shell microparticle is subjected to is high intensity focused ultrasound (HIFU).

Typically, the frequency of the radiation of the pressure wave the microparticle is subjected to is 10 MHz or less. Typically, the frequency of the radiation of pressure wave the microparticle is subjected to is 0.25 MHz or greater. Lower frequency radiation can penetrate deeper than high frequency radiation and can therefore reach disease sites at further distances from the surface of the skin. This can be desirable in the case of the present invention, where diseased sites may be found relatively deep under the skin. However, it is important that the frequency is not so low as to be in the region of infrared radiation, which can be damaging to biological tissue.

Thus, typically, the frequency of radiation which is used to embed the microparticle of the invention is 5 MHz or less, preferably 2.5 MHz or less, preferably 2 MHz or less. Typically, the frequency of radiation which microparticles of the invention are subjected to is 0.1 MHz or higher, preferably 0.25 MHz or higher. Preferably the frequency of radiation is from 0.25 MHz to 10 MHz, preferably from 0.25 MHz to 5 MHz, more preferably from 0.25 MHz to 2.5 MHz, most preferably from 0.25 MHz to 2 MHz.

Typically, the pressure of the radiation which is used to embed the microparticle of the invention is 10 MPa or less, preferably 8 MPa or less, preferably 6 MPa or less, more preferably 4 MPa or less. Typically, the pressure of the radiation is 0.1 MPa or higher, preferably 0.5 MPa or higher, preferably 1 MPa or higher, more preferably 2 MPa or higher. Preferably the pressure of the radiation is from 2 MPa to 4 MPa.

In some embodiments of the invention, the microparticle may be subjected to ultrasound imaging after administration. This can allow the particle to be imaged to ensure it has reached and/or is successfully embedded in the diseased site.

Having multiple surface cavities reduces the threshold to nucleate cavitation for the gas bubbles of the microparticle of the invention and it has surprisingly been found that the particle described herein exhibits stable cavitation. The particle of the invention has an advantage of sustained cavitation for extended durations compared to that of microbubbles. The microparticle of the invention has a low cavitation threshold, exhibits stable cavitation for extended time periods, and can be imaged using ultrasound at low MI. A low cavitation threshold and extended stable cavitation reduces time constraints on ultrasound imaging techniques which would otherwise have to be performed quickly under time pressure before inertial cavitation occurs.

In the invention, the ultrasound imaging typically occurs at a mechanical index (MI) of 2 or less. The MI of radiation is a measure of the power of an ultrasound beam, designed as an indication of the potential for harmful, non-thermal effects on the body from the beam. Currently, the FDA stipulates that the MI of ultrasound scanners must not exceed 1.9 for diagnostic imaging, with values much below this maximum threshold being preferred. The multi-cavities of the invention lower the MI threshold that is needed to nucleate cavitation. Therefore imaging can occur at a low MI. This is highly advantageous as it reduces risk to the body associated with exposure to ultrasound

In the invention, the ultrasound imaging occurs at a mechanical index of 2 or less, preferably 1.8 or less, preferably 1.6 or less, preferably 1.4 or less, preferably 1.2 or less.

Typically, where the microparticle of the invention is imaged using ultrasound, it preferably has an average diameter of from 5 to 10 µm, preferably from 5 to 6 µm.

For ultrasound imaging it is generally preferably to have higher frequency radiation as this generally provides a higher resolution image. However, at too high a frequency, the radiation may not penetrate deep enough to image particles at the diseased site. Therefore, typically, the frequency of radiation which a microparticle of the invention is subjected to when undergoing ultrasound imaging is 30 MHz or less, preferably 20 MHz or less, preferably 15 MHz or less. Typically, the frequency of radiation which a microparticle of the invention is subjected to is 5 MHz or higher, preferably 10 MHz or higher. Preferably the frequency of radiation which a microparticle of the invention is subjected to is from 5 MHz to 30 MHz, more preferably 10 MHz to 15 MHz.

### EXAMPLES

### Example 1: Preparation and Characterisation of PLGA Microparticles

### Preparation

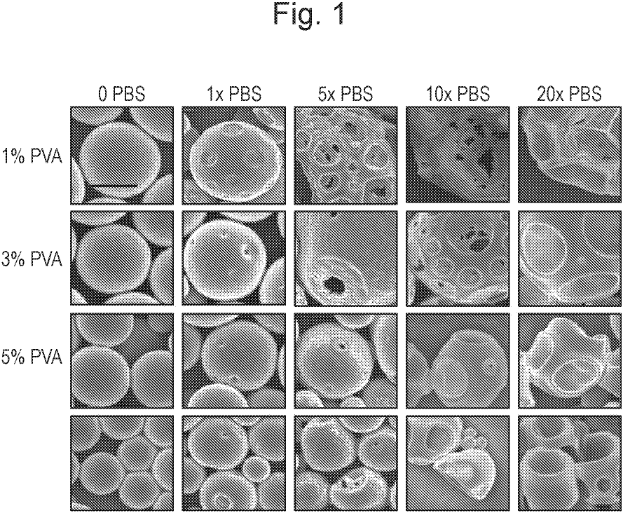

Multi-cavity PLGA microparticles (mcPLGA MPs) were prepared by an adapted water/organic/water double emulsion solvent evaporation process. 50 mg of poly(lactic-co-glycolic acid) PLGA was dissolved in 2 mL of dichloromethane (DCM). Then 100 µl of phosphate buffered saline (PBS) was added to the PLGA solution and sonicated (Ultrasonic processor VCX 130, Sonics and Materials Inc., USA) at 100 W for 30 s in an ice bath to form an emulsion. The obtained water-in-oil (W/O) emulsion was poured into a 5% poly(vinyl alcohol) (PVA) solution and homogenized (Ultra Turrax T-25 Ika Labortechnik, Germany) at 12000 rpm over ice for 5 min. Then this particle suspension was stirred at room temperature for 3 h in a chemical fume hood to allow for evaporation of the organic solvent. The PLGA particles were collected by centrifugation at 1,000 G for 5 min, after which they were redispersed and then subjected to three cycles of centrifugation/wash/redispersion. After the final wash, the fresh microparticles were frozen at - 80° C. and then lyophilised in a lyophiliser (Alpha 2-4 LSCbasic, Christ, Germany) for 48 h to achieve a dried powder for long term storage. For drug loaded PLGA, therapeutics was added to the organic solution prior to emulsification. The resulting particles are shown in FIG. 1, and show a broad range of shapes (from smooth to porous) and sizes (from 0.6 µm to 6 µm in diameter), synthesized under different concentrations of PBS and PVA. Alternative formulations investigated using the same method were 0x, 1x, 5x, and 10x PBS, and 1%, 3%, 5%, and 10% PVA. 1x PBS is given as 0.01 M concentration in accordance to the manufacture instructions.

### Rhodamine B (RhB) as a Model Drug for HIFU Enhanced Drug Delivery Study

The model drug, rhodamine B (RhB), was encapsulated in mcPLGA MPs (RhB-mcPLGA MPs) using emulsion solvent evaporation technique mentioned above. The quantity of RhB present was calculated according to the UV-absorbance of RhB at 553 nm measured by a UV-Vis Spectrometer (Shimadzu UV 2450). A standards curve was made in PBS to correlate the mass of RhB in solution with the UV-absorbance spectral curve. The loading efficiency was calculated by first measuring the remaining RhB within in the supernatant of RhB-mcPLGA MPs after solvent evaporation and subtracting it from the total amount of RhB added into the system. This difference was divided by the total amount of RhB added and multiplied by 100 to obtain the percent of RhB loaded.

### Characterisation Technique

Size and surface morphology of the resultant RhB-PLGA particles were assessed using a JEOL JSM-6700 Field Emission Scanning Electron Microscope (FE-SEM; JEOL Ltd., Akishima, Tokyo, Japan) at an acceleration voltage of 5 kV. Samples for the SEM were prepared by dropping 10 µl of 1 mg/ml suspension on silica wafers and air drying. The wafers were mounted onto a metal stub using double-sided electrical tape and coated with platinum (JFC 1600 Auto Fine Coater, JEOL Ltd., Akishima, Tokyo, Japan) for 2 min at 20 mA. All images were recorded under Secondary Electron Imaging (SEI) mode. Size distributions were determined by dynamic light scattering (DLS) (Malvern Nano-ZS, Malvern Panalytical, Malvern, UK).

### Characterisation Results

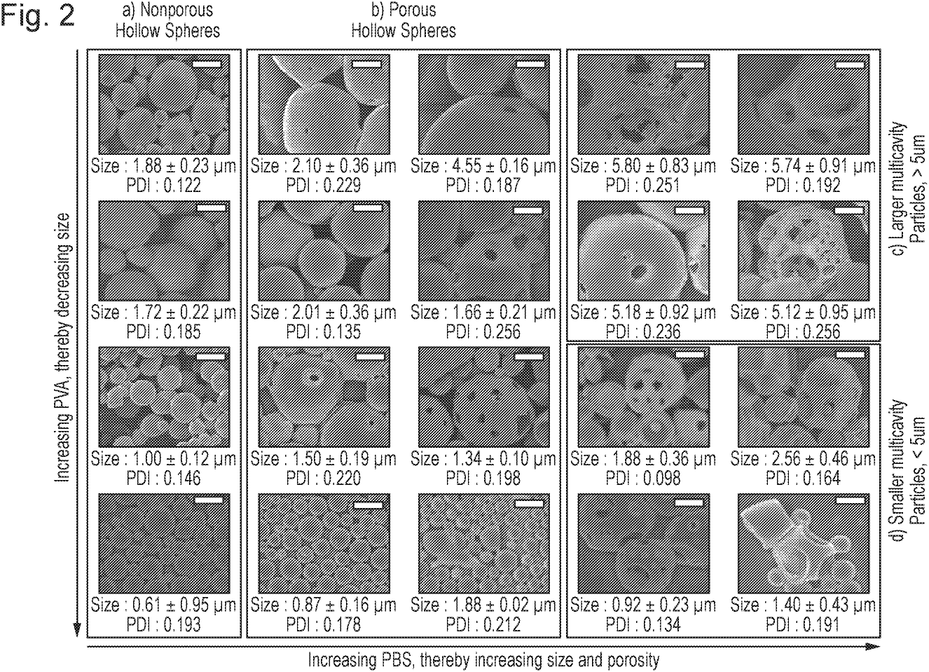

SEM images of PLGA microparticles (FIG. 2) prepared using the double emulsion-diffusion-evaporation method exhibited a multitude of diameters and porosity proportional to the concentration of porosigen (PBS) and stabilizer (PVA) and are been labelled into 4 categories, details of which are provided in the following sections, based on their morphology and diameter namely a) non-porous hollow spheres, b) porous hollow spheres, c) large multi-cavity particles (> 5 µm in diameter), and d) small multi-cavity particles (<5 µm in diameter). For ease of understanding, the type of pore openings were classified either as pores (holes running throughout the thickness) or as cavities (surface indentations not running throughout the thickness of the shell but limited to the surface).

PLGA microparticle formulations without PBS in the internal aqueous phase led to nonporous hollow spheres irrespective of the quantity of stabiliser. Increasing the amount of porosigen in the internal aqueous phase of the water-in-oil-in-water (W/O/W) droplet resulted in hollow spheres with small and infrequent pores. Further increases of PBS concentration led to multi-cavity particles that were not uniformly spherical. Instead, cup shapes, highly porous spheres, and various aspherical shapes were present. In contrast, increasing the amount of stabilizer present in the bulk aqueous phase prior to heating inhibited the presence of pores and decreased the diameter of the polymer particles for all formulations. The population of multi-cavity particles may have porous particles present and vice versa. Thus, these categories are based on the predominant observed structure.

For this study, the multi-cavity particles were separated into two groups based on diameter, i.e., large multi-cavity particles (> 5 µm) and small multi-cavity particles (< 5 µm). This cut-off to distinguish the larger from the smaller variants at 5 µm was chosen so as to distinguish the multi-cavity particles and compare their acoustic response to both setups of HIFU and the diagnostic imaging and then determine their ideal potential use in the different ultrasound regimens. Most commercially available ultrasound contrast agents (UCAs) have a diameter of less than 5 µm in diameter on average, so this enables a comparison to be made with commercially available UCAs, and also enables a study of the different performance of the larger particles. Smaller variants would be ideal for use in the therapeutic domain given their favourable size, and the larger particles would nucleate bubbles with a larger scattering cross section and behave as better contrast agents but achieve poorer perfusion.

### Example 2 - Model Drug Delivery

### HIFU Setup

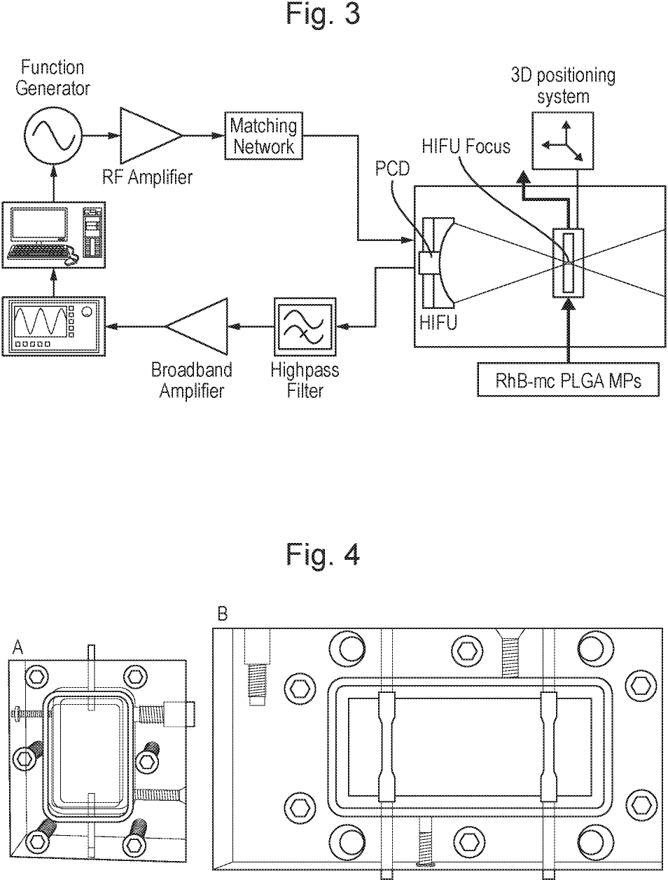

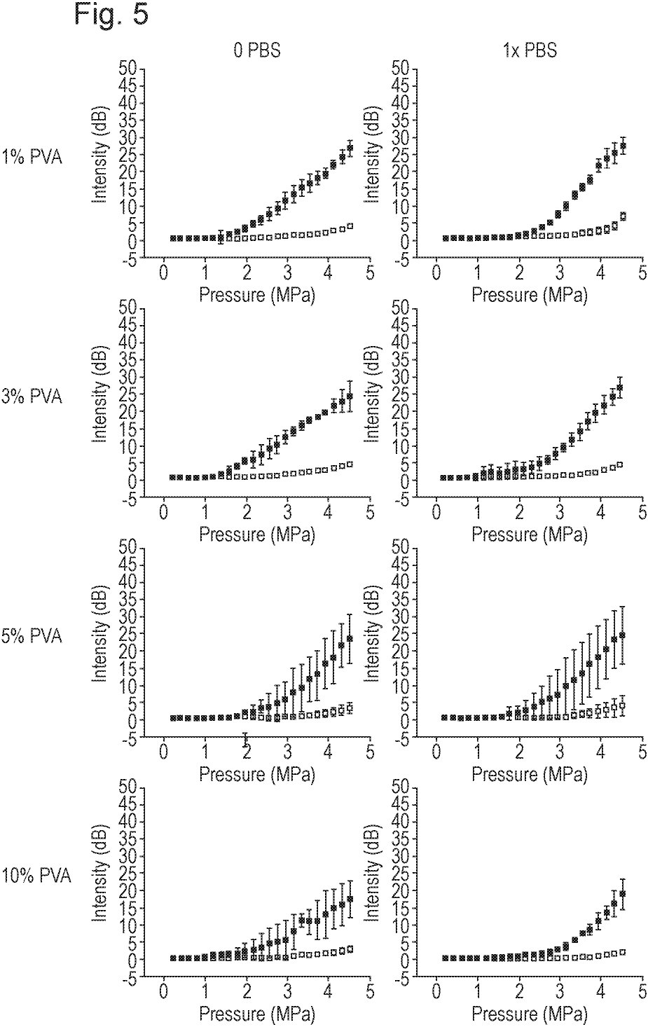

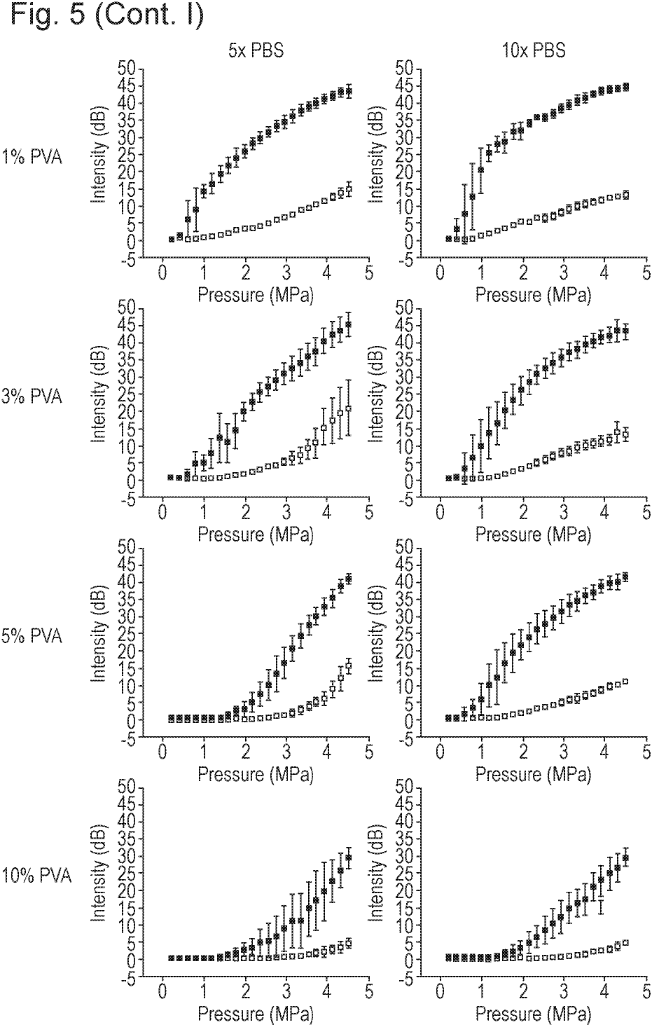

Using a conventional high intensity focused ultrasound (HIFU) setup (FIGS. 3 and 4), the particles of example 1 were exposed to 1.1 MHz ultrasound at various pressure amplitudes. FIG. 5 shows the cavitation intensity (i.e., the likelihood for the suspension of particles to respond to ultrasound) and indicates that the particles respond to HIFU at drastically lower pressure amplitudes as compared to water and spherical variants (following the same production method). These pressure amplitudes are comparable to, if not lower, than those of other polymeric cavitation nucleation agents currently under investigation in other groups.

### HIFU Enhanced Drug Delivery

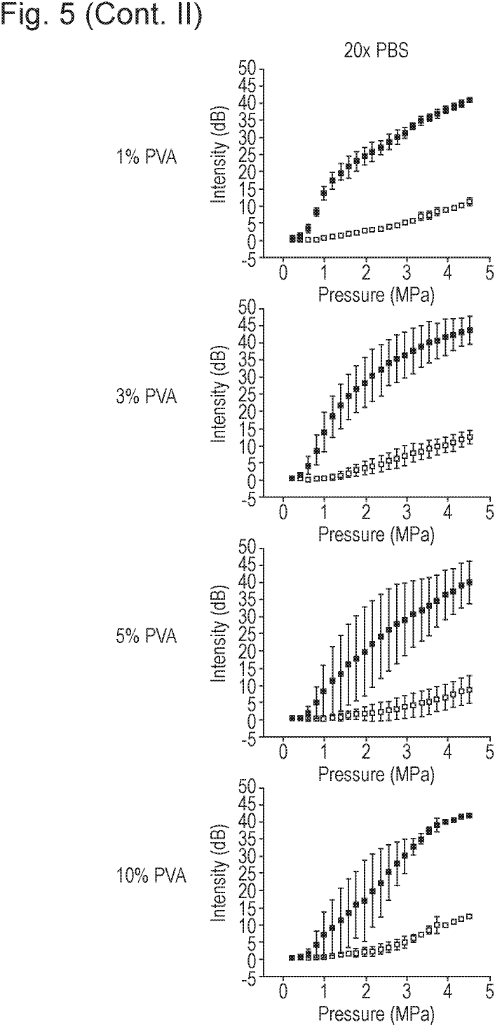

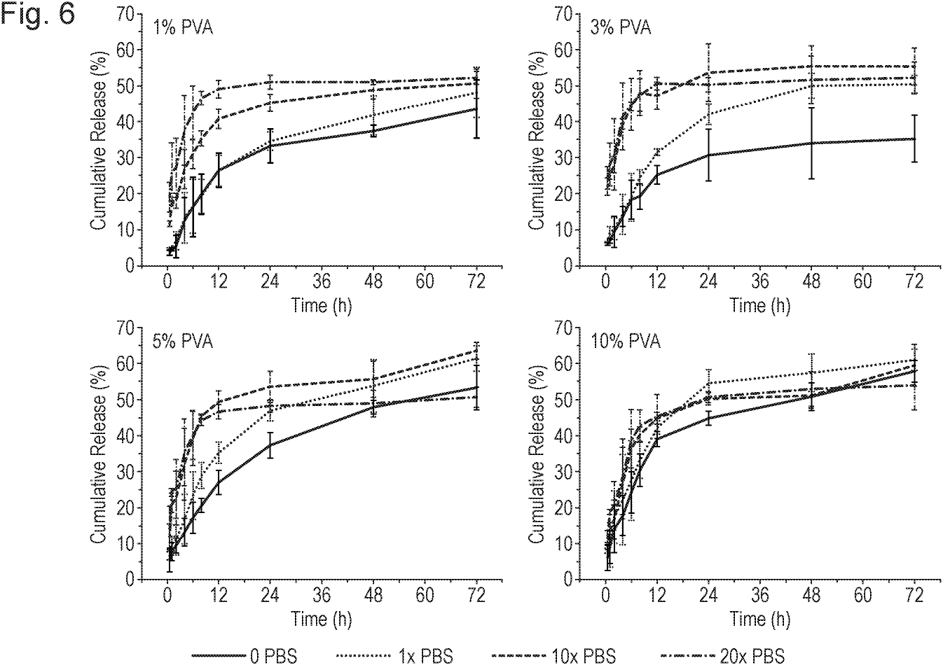

The in vitro release study was performed in PBS buffer solutions using sample and separation method. 50 mg of freeze-dried PLGA/rhodamine was collected and dispersed in 50 ml of 0.01 M PBS (pH 7.4) buffer solution in sealed vials. This solution was maintained at 37° C. under magnetic agitation. At each time point, 1 ml of the solution was taken out and centrifuged at 3000 RCF for 5 min. The concentration of the drug in the collected supernatant was analyzed using UV-visible spectrophotometer at 553 nm. The experiment was performed in triplicate. Results (FIG. 6) show a rapid release of the model drug in 24 hours, likely due to the edges of the cavities dissolving first (FIG. 7). Afterwards, the model drug was slowly released across two weeks.

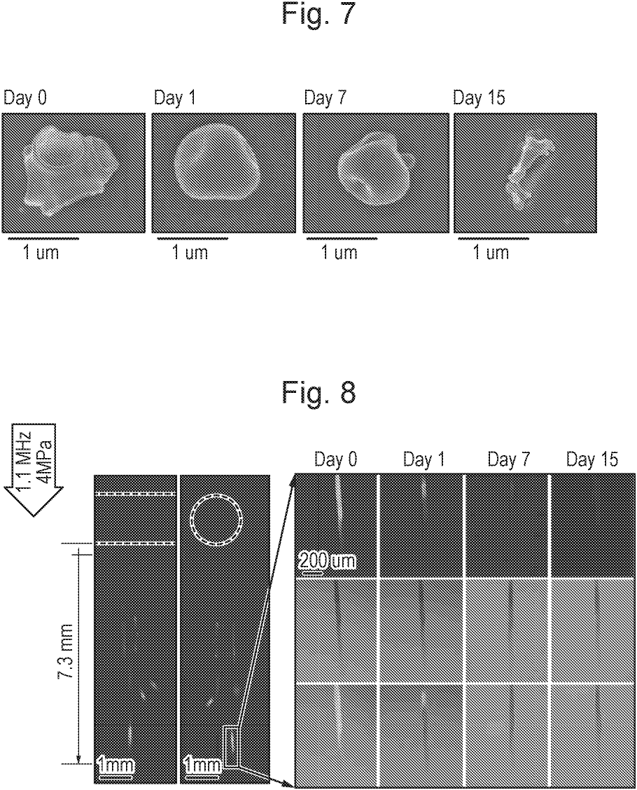

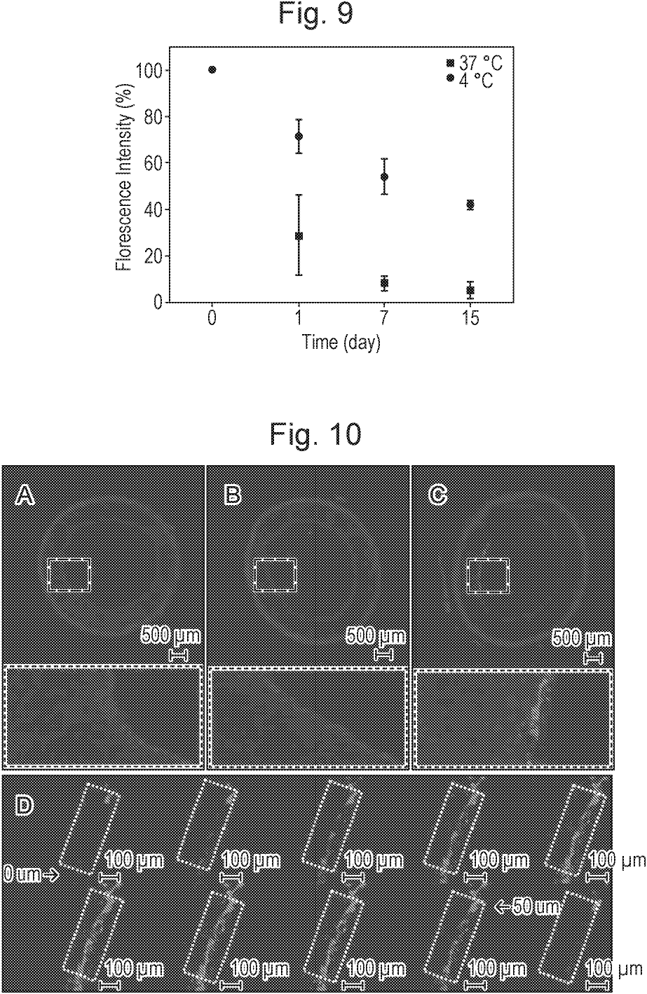

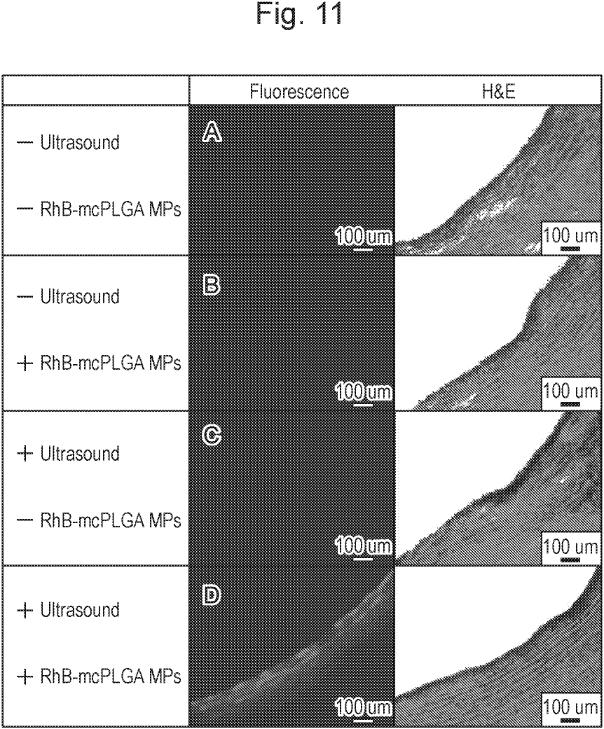

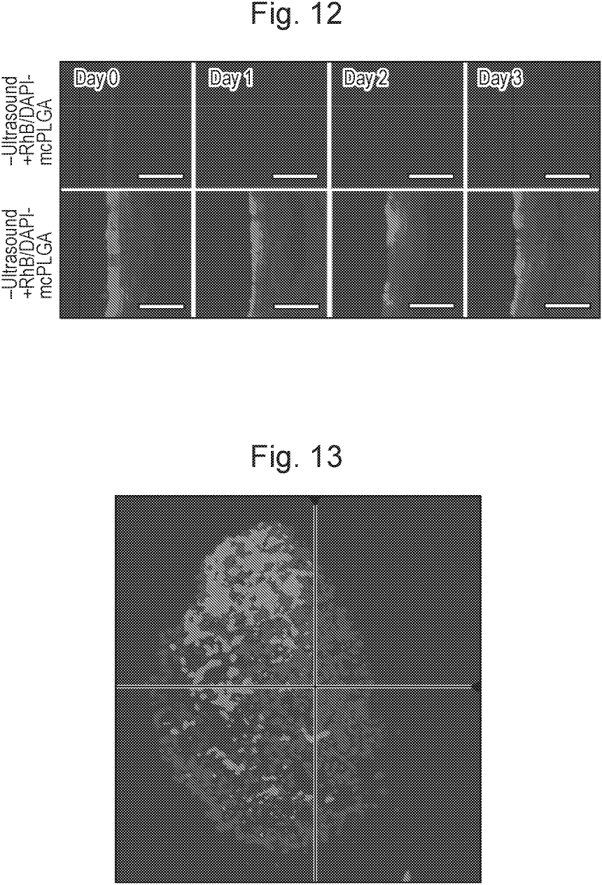

The RhB-mcPLGA MPs were then tested to determine the capability to be embedded into an agarose model. FIG. 8 shows that the particles were implanted into the agarose at depths of up to 7 mm. The particles remained in the agarose for 15 days, slowly releasing the fluorescent dye. This was quantified at both 37 C and 4 C (FIG. 9). Similar experiments were conducted with porcine arteries. FIG. 10 shows that particles were able to penetrate between the intima and media of the artery and remain embedded. Only the region that was targeted shows signs of mcPLGA MPs implantation, suggesting this method is spatially controllable. Furthermore, these particles are not simply on the surface of the slides and are seen throughout the thickness of the targeted area. The process also does not further damage the endothelium (FIG. 11).

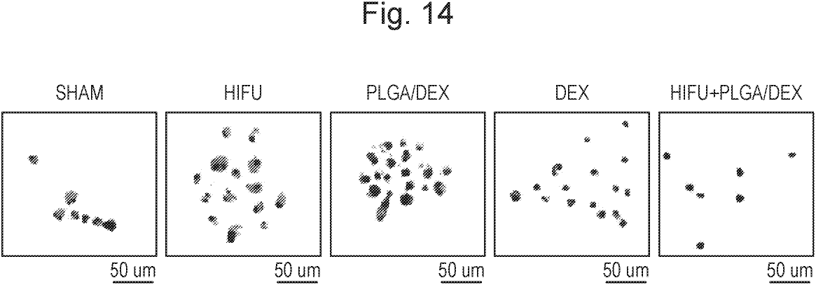

We have also loaded the mcPLGA MPs with both DAPI and RhB, and remotely implanted the loaded particles using HIFU into ex vivo porcine arterial tissue. We monitored the location of both the DAPI and RhB across three days (FIG. 12). Here, RhB labels the location of the particles, whereas DAPI is only fluorescent when bound the DNA. Therefore, DAPI was only observed after release from the particles during degradation, whereby the molecule diffused across the cellular membrane and bound to the DNA of the cell. With each day, DAPI was found to travel further from the initial implantation site.

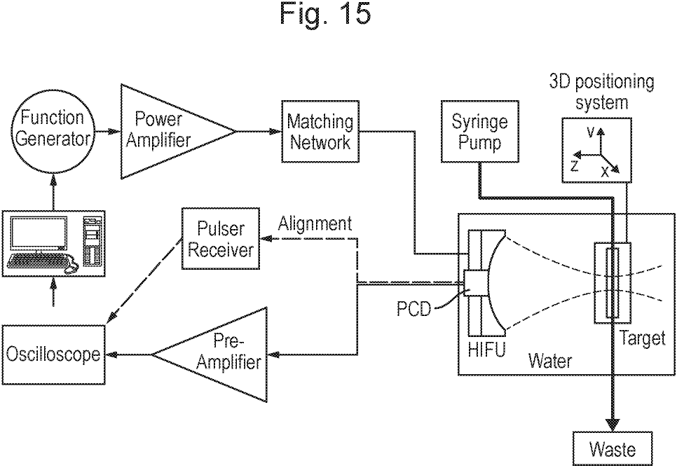

As the cells in the ex vivo porcine artery were considered non-viable, we have also tested the ability to deliver DAPI to viable cells using a 3D foam cell spheroid model (FIG. 13). Similar to the porcine arteries, the DAPI diffused through the membranes of the cells to stain the DNA of the foam cells. This shows the capability to deliver therapeutics to the cells without damaging the entire structure or integrity of the cell mass.

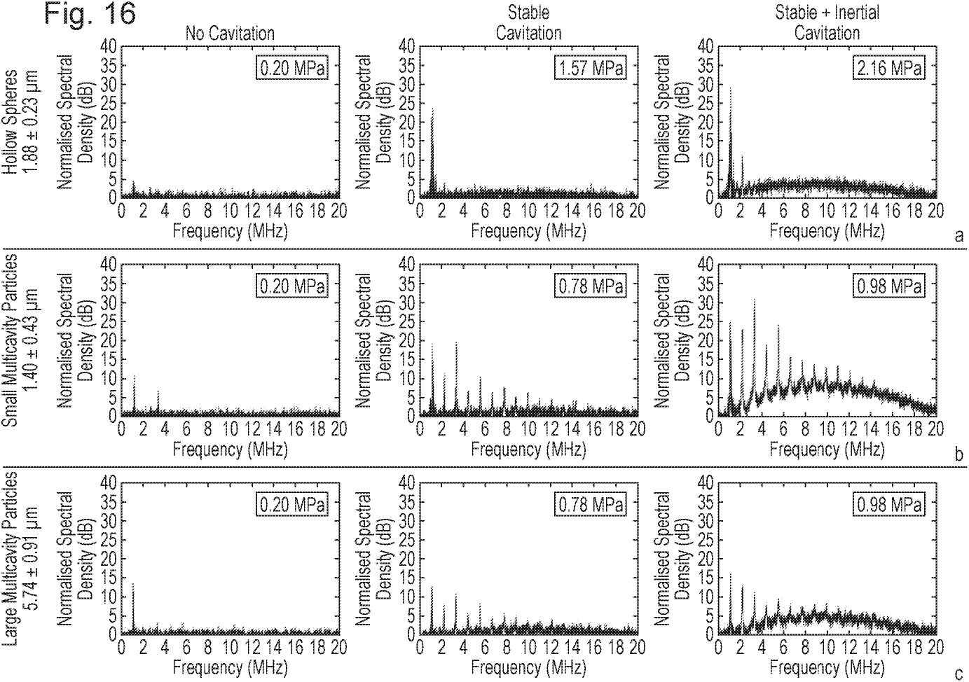

Furthermore, we have also been able to load dexamethasone (Dex) into the mcPLGA MPs and test efficacy of Dex-PLGA MPs in reducing lipid accumulation in foam cell spheroids. To determine the physiological effects of Dex on cholesterol loaded foam cell spheroids, the Oil Red O stain was used to determine the presence of neutral lipids and triglycerides in cells. Lipid was stained in red and was visualised through an optical microscope (FIG. 14). HIFU implanted Dex-PLGA show to be more effective in reducing lipid accumulation.

### Example 3 - Acoustic Characterisation of Microparticles

### Sample Chamber

To make the acoustically transparent agarose sample chamber, 1% (w/v) of agarose solution was boiled and degassed for 30 min. The agarose solution was then poured into a bespoke cuboid mould (50 mm in length × 30 mm in width) and sealed with acoustically transparent mylar windows. A 1.6 mm steel rod was threaded through the mould. The rod was removed after gelation was complete, creating a channel for fluid flow.

### Resuspension of Microparticles

The dry powder, as prepared in Example 1, was resuspended by mixing with deionized water and vortexed briefly (<5 sec) which led to no agglomeration as indicated by a the polydispersity index (PDI) = 0.13 by DLS. The particles were pumped into the acoustic sampling chamber and the containment vessel and no sedimentation was observed for particle diameters < 5 µm. For larger variants, i.e. diameter > 5 µm, sedimentation was observed after 10 min. To prevent this, all experiments were completed within 7 minutes.

### Therapeutic Ultrasound Setup

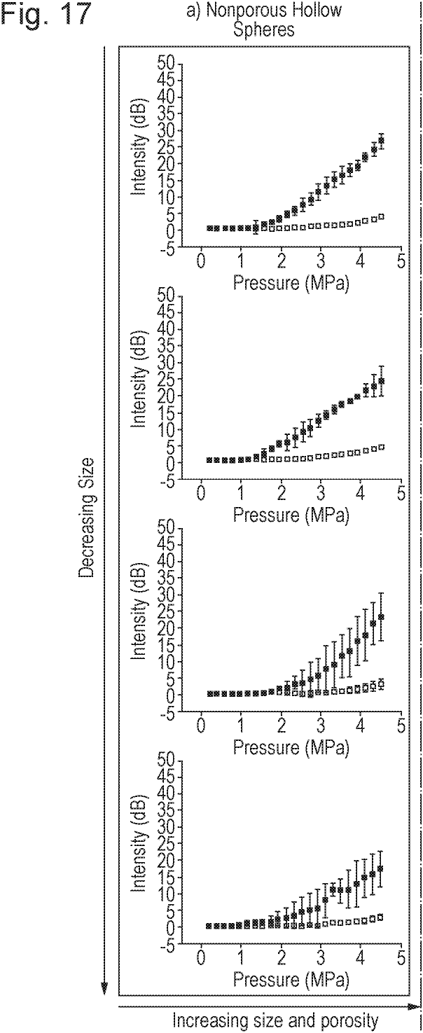

A 1.1 MHz high intensity focused ultrasound (HIFU) transducer (H102, Sonic Concepts, Bothell, WA, USA) was used for acoustic excitation. A 15 MHz passive cavitation detector (PCD) (V319, Olympus, Singapore) - co-axially aligned with the HIFU transducer focus - was used for detection of acoustic emissions at the HIFU focus. The HIFU transducer was calibrated using a 0.2 mm needle hydrophone (SN2562, Precision Acoustics, Dorset, UK). The geometric focus of the transducer was 1.37 mm in width and 10.21 mm in length. The HIFU transducer was driven by a function generator (33210 A, Keysight Technologies, Santa Rosa, CA, USA) and a RF power amplifier (1040 L, Electronics & Innovation, Rochester, NY, USA). All experiments with HIFU were carried out in a large tank filled with filtered, degassed, and deionized water. Acoustic amplitudes in this study were reported in MPa peak negative pressure amplitudes. A schematic representation of the setup is shown in FIG. 15.

### Acoustic Characterization of Microparticles

The agarose phantom sample chamber was submerged in the degassed water tank and aligned to the focus of the transducer. With the channel filled with air, the PCD was driven with a pulser-receiver (JSR Ultrasonics DPR300, Imaginant, Pittsford, NY, USA) to determine the position of the channel. A 3D positioning system was used to adjust the chamber until the channel was at the focus of the HIFU transducer. A 1 mg/ml suspension of microparticles were flowed through the channel using a syringe pump at a rate of 0.2 ml/min for ultrasound exposures. PLGA microparticles were exposed to 20 cycle bursts with increasing peak negative pressure amplitude at a pulse repetition period of 0.1 s. Acoustic emissions from PLGA microparticles were detected using a 15 MHz PCD co-axially aligned with the HIFU transducer. The PCD output was amplified using a broadband preamplifier (SR445A, Stanford Research Systems, Sunnyvale, CA, USA). The received signals were then recorded onto an oscilloscope (DXOX3032A, Keysight Technologies, Santa Rosa, CA, USA) and post processed to determine the power spectral density (PSD) curve. For each burst, the area under the PSD curve was determined and compared to degassed water exposed to HIFU under the same conditions. Following the signal processing using MATLAB R2019b cavitation was considered to have occurred if the received signals were 6 dB higher than noise from the water control. The probability of cavitation was determined as the percentage of bursts that recorded a cavitation event out of the total number of HIFU bursts (120 bursts).

### Cavitation Threshold Determination

To estimate the cavitation threshold, a sigmoid function was fit to the probability for both harmonic and broadband signal. The sigmoid fitting function is defined in eq. 1:

$$f = \frac{1}{1 + {e}^{- \left({\left(p - {p}_{50}\right)}^{k}\right)}}$$

Where, f is the probability for cavitation, p is the input pressure, p₅₀ is the cavitation threshold defined as the pressure amplitude value for achieving in 50% of the total number of pulses contained a cavitation, k is the slope of the fit. This function was fit to the experimental data by minimising the sum of square residuals using Microsoft Excel.

### Cavitation Response

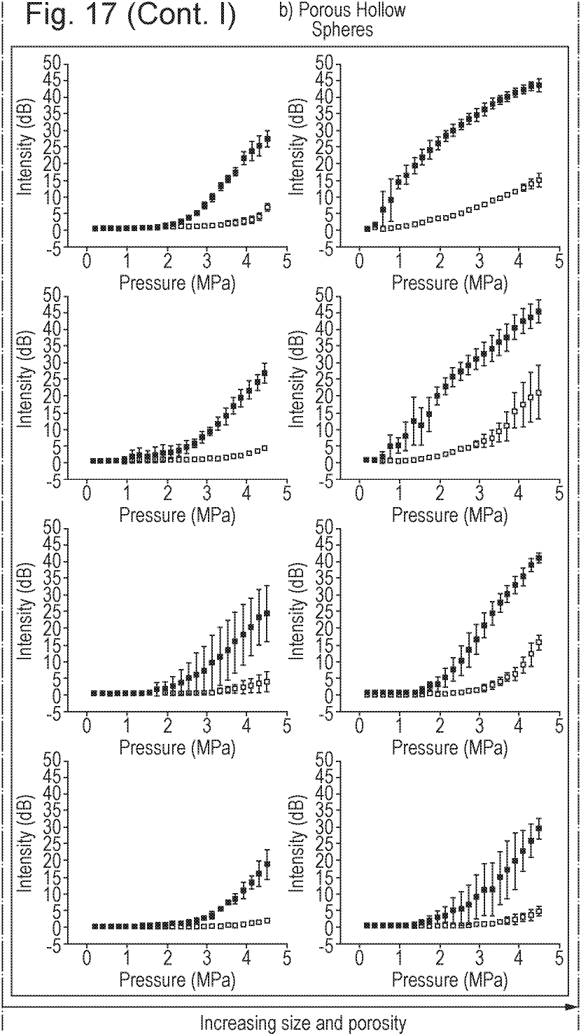

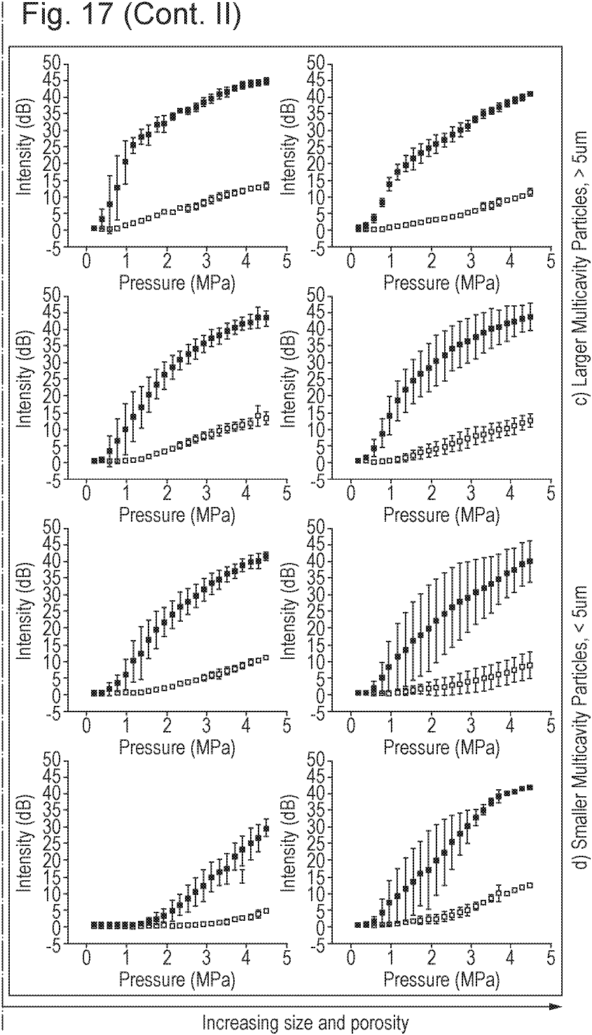

FIG. 16 shows representative images of normalized PSD curves for three different shapes of particles, namely the hollow spheres, small multi-cavity, and large multi-cavity microparticles. Cavitation was detected for all types of microparticles. Although the presence of harmonic emissions was observed for all microparticle formulations, substantial broadband emissions were only present for some of the formulations and was dependent on both the diameter of the microparticle and acoustic intensity (FIG. 17). Broadband emissions, if present, only became apparent at pressure amplitudes larger than the pressure amplitudes required for harmonic emissions.

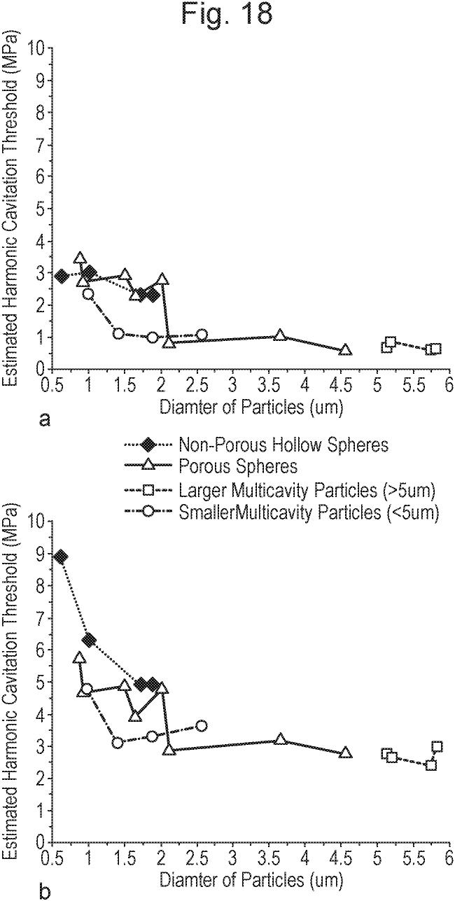

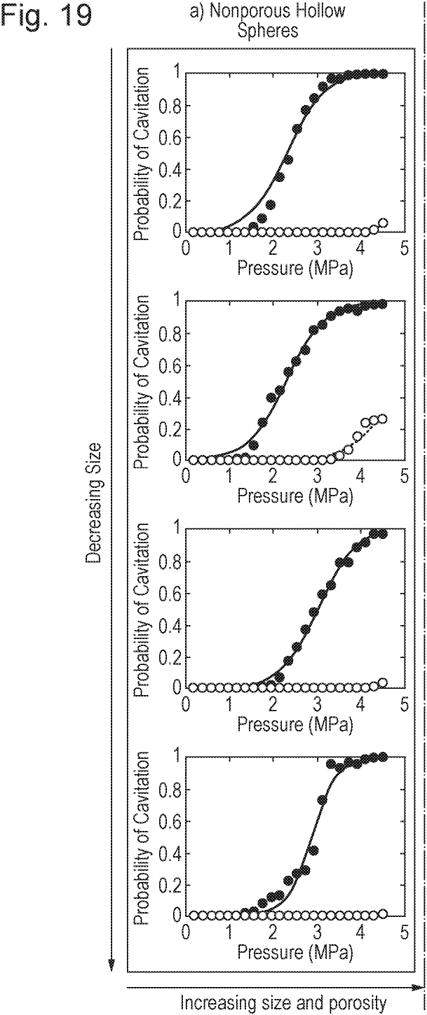

FIG. 18 shows the estimated harmonic and broadband cavitation thresholds determined by the probability of cavitation (FIG. 19) for all the microparticles tested. Both harmonic and broadband thresholds were governed by the diameter and shape of the microparticles. Irrespective of shape, larger microparticles had lower cavitation thresholds. This trend was most evident for the onset of broadband noise. Regarding the shape of the microparticles, there was generally a lower cavitation threshold for both harmonic and broadband emissions for porous particles compared to smooth hollow spheres. Similarly, more porous particles, i.e., multi-cavity microparticles as opposed to surface pores on spheres, emitted harmonic and broadband noise at lower input pressures; larger cavities nucleated cavitation at the lowest acoustic intensity.

Our results for the inertial cavitation threshold for the hollow spheres indicated that a pressure range between 4.5 MPa to 9 MPa was required to achieve 50% probability of inertial cavitation. As the size decreased for each morphology group, a higher input pressure was required to achieve 50% probability of harmonic cavitation.

### Example 4: Contrast Enhancement

### Diagnostic Acoustic Set-Up

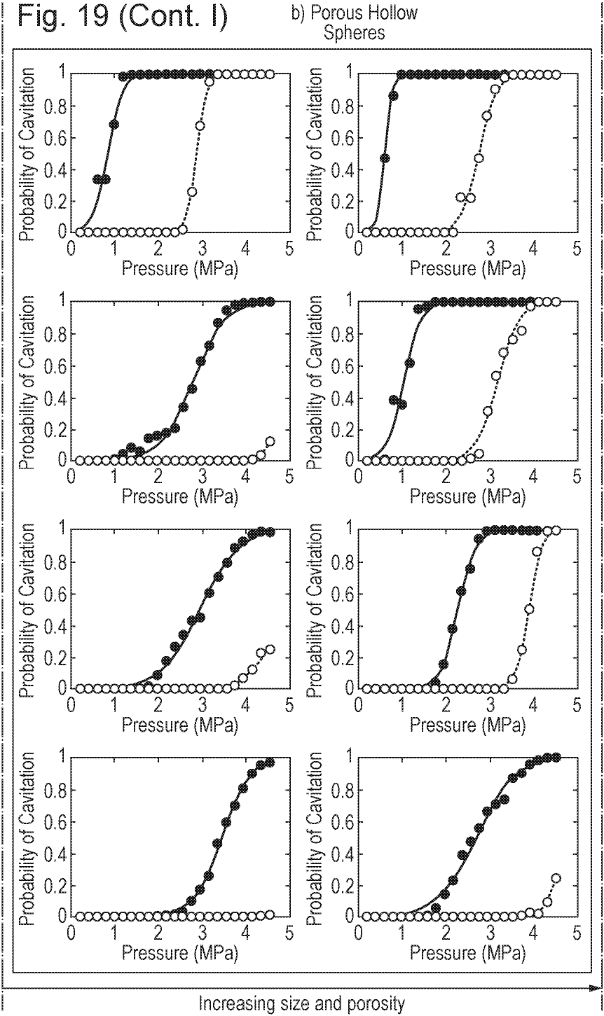

An E-Cube 12-R (Alpinion Medical Systems, Seoul, South Korea) clinical ultrasound imaging system with a linear array transducer (L3-12, Alpinion Medical Systems, Seoul, South Korea) was used to acquire images at the focal zone depth (5 cm) at a 12 Hz framerate. Scanning was performed with B mode operating at 10 MHz. Additionally, the mechanical index of this scanner was 1.1 at a 100% acoustic power giving a peak negative pressure of 3.2 MPa 99. Data was saved in triplicate for each sample. Three independent samples for each formulation were tested. A schematic representation of the diagnostic ultrasound setup is shown in FIG. 20a.

### Contrast Enhancement Measurements

For contrast enhancement measurements, a flow system was implemented using an acrylic water bath, a syringe pump (KD Scientific, Holliston, MA, USA), and flexible low-density polyethylene tubes (outer diameter 2.42 mm, thickness 0.37 mm). A dose of 6 mL reconstituted PLGA particles (1 mg/ml) were infused into the phantom holder via a syringe pump at a constant rate of 1 ml/min. The sample holder was placed in a water bath and the probe was placed directly above the vessel. As a control, deionized water was also run through the sample chamber and saved. The data was saved in triplicate in B-Mode. Afterwards contrast to tissue ratio (CTR) analysis was performed using ImageJ 1.52q (National Institutes of Health, Bethesda, MD, USA) to quantify the ability of each PLGA particle sample to distinguish between vessel and tissue using eq 2:

$$C T R = \frac{2 {\left({μ}_{t} - {μ}_{v}\right)}^{2}}{{\left({σ}_{t}^{2} - {σ}_{v}^{2}\right)}^{2}}$$

where µₜ and µᵥ represent the mean backscatter signal strength in the tissue and within the vessel lumen region, respectively, while σₜ², and σᵥ² represent the corresponding variances. Four region-of-interests (ROIs) within the tissue and two ROIs within the vessel were selected. Each ROI was a 0.5×0.5 mm square. Images were acquired in triplicate for each sample using the linear array probe. The mean signal was averaged across all tissue and vessel ROIs to reduce variability. The four tissue ROIs were selected along the same horizontal and vertical axes as the vessel ROIs (as shown in FIG. 20b).

### Contrast Enhancement Results

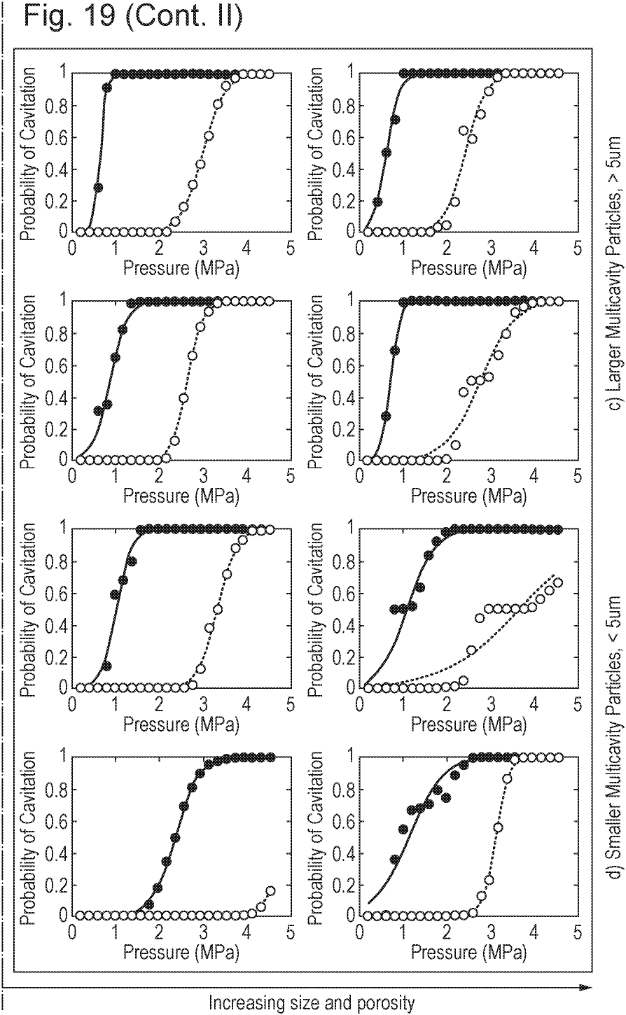

The representative images for all the microparticle formulation samples tested with the diagnostic ultrasound setup (as described above) at maximum input power, along with their calculated CTR values are shown in FIG. 21. Generally, smaller and smoother microparticles had lower CTR values compared to more porous particles. The highest CTR values corresponded with larger multi-cavity particles (5.12 µm to 5.18 µm in diameter).

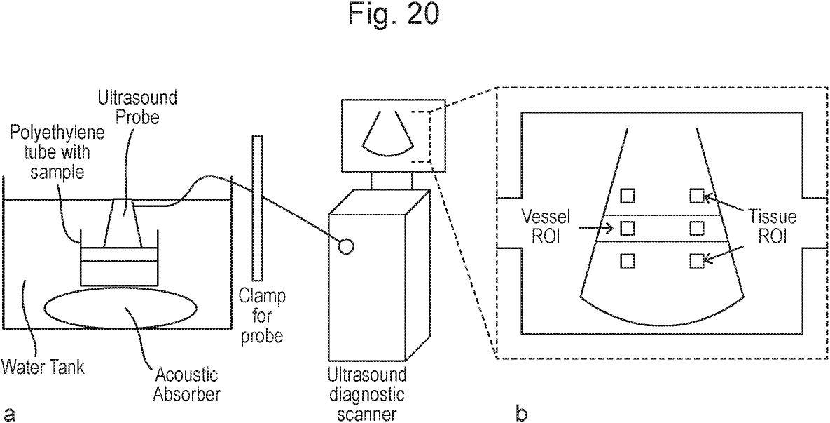

FIG. 22 shows the measured CTR for representative microparticles from the different morphology groups (2 µm in diameter smooth spheres, 2 µm in diameter multi-cavity microparticles, and 6 µm in diameter multi-cavity microparticles) in addition to deionized water for increasing input pressures from 10% to 100% power (corresponding MI values of 0.11 to 1.1). The CTR of the 2 µm in diameter smooth spheres remained at 6 dB for all input powers tested. Smaller multi-cavity microparticles provided CTR values greater than the smooth spheres at all input powers and displayed a subtle increase in CTR for input powers greater that 40%. Larger multi-cavity microparticles consistently delivered the highest CTR values for all powers tested. Similar to the smaller multi-cavity particles but to a greater extent, the CTR of the larger multi-cavity particles increased with increasing input power.

For all PLGA microparticle formulations tested to emit detectable harmonic noise in response to therapeutic ultrasound, was unexpected. Such results are indicative of stable cavitation.

### Example 5 - Contrast Enhancement of Model Drug Delivery Particles

### Preparation

As in Example 4, an E-Cube 12-R (Alpinion) with a L3-12 transducer was used to acquire images at the focal zone depth (5 cm) at a 12 Hz framerate. Scanning was performed with B mode operating at 8.5 MHz. Additionally, the mechanical index of this scanner was 1.1 giving a peak negative pressure of 0.47 MPa.

An acoustically transparent agarose sample chamber was made from a 3% (w/v) of agarose solution, which was boiled and degassed for 30 min to prevent cavitation as a result of endogenous bubbles. The agarose solution was then poured into a bespoke cuboid mold (50 mm in length × 30 mm in width) and sealed with acoustically transparent mylar windows. A 1.6 mm steel rod was threaded through the mold. After gelation was completed, the rod was removed, creating a flow channel.

### Data Collection

For this, a flow system was implemented using an acrylic water bath, a KD Scientific syringe pump (MA, USA), and flexible PVC tubes. A dose of 6 mL reconstituted PLGA particles were infused into the phantom holder via a syringe pump at a constant rate of 1ml/min. The sample holder was placed in a water bath and the probe was placed directly above the vessel. All samples were tested at a concentration of 1 mg/ml in this setup. The acoustic power was set at 60% as prior results showed highest enhancement at 60% power for test samples. Images were saved in triplicate for each sample. As a control, deionised water was also run through the sample chamber and saved.

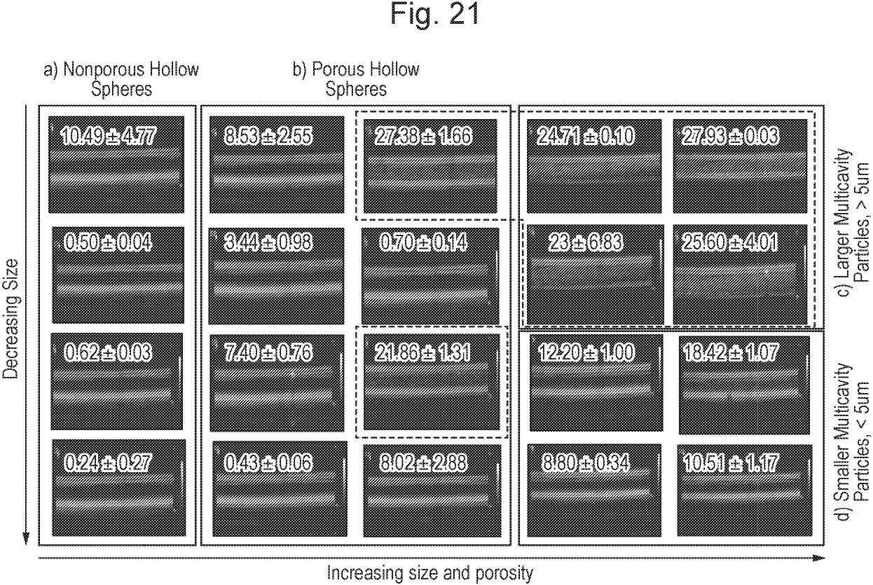

As in Example 4, the data was saved in triplicate in B-Mode and save as beamformed data. Afterwards contrast to tissue ratio (CTR) analysis was performed to quantify the ability of each PLGA particle sample to distinguish between vessel and tissue using the following equation (eq. 2):

$$C T R = \frac{2 {\left(μ t - μ v\right)}^{2}}{{\left(σ {t}^{\hat{}} 2 - σ {v}^{\hat{}} 2\right)}^{2}}$$

where µₜ and µᵥ represent the mean backscatter signal strength in the tissue and within the vessel lumen region, respectively, while σₜ², and σᵥ² represent the corresponding variances. Four region-of-interests (ROIs) within the tissue and two ROIs within the vessel were selected. Each ROI was a 1x1 mm square. Images were acquired in triplicate for each waveform at the optimized acoustic output using the linear probe. The mean signal was averaged across all tissue and vessel ROIs to reduce variability. The four tissue ROIs were selected along the same horizontal and vertical axes as the vessel ROIs (as shown in FIG. 23)

[image]

### Results

The results for the CTR analysis and representative images are shown in FIG. 23. As can be seen and as expected, highest CTR values were obtained for lesser PVA and higher PBS concentrations. Increasing PVA decreases the size and increasing PBS increases the porosity of these particles, thereby increasing the number of cavities on the surface. CTR values as high as 49 dB were achieved, although with relatively larger particles (diameter > 5 um; 1% PVA 10× PBS). However, the clinically relevant particle sizes (diameter<2 um) were 5% PVA 10× PBS having a CTR of 13.94 dB. These particles achieved enhancement comparable to the commercially available ultrasound contrast agents (CTR values 10-20 dB). These results indicate that these particles are capable of providing contrast enhancement required for clinical imaging and can be used as both drug delivery agents and contrast agents.

### Example 6: Preparation and Characterization of Microparticles

### Preparation of Drug-Loaded mcPLGA Microparticles (MPs)

mcPLGA microparticles were prepared as described in Example 1, except in that 0.5 mg of RhB was dissolved with the PLGA. For the fabrication of Dex loaded mcPLGA MPs, 0.5 mg of the payload was added before sonication and homogenization following the method described of Example 1.

### Characterization of mcPLGA MPs

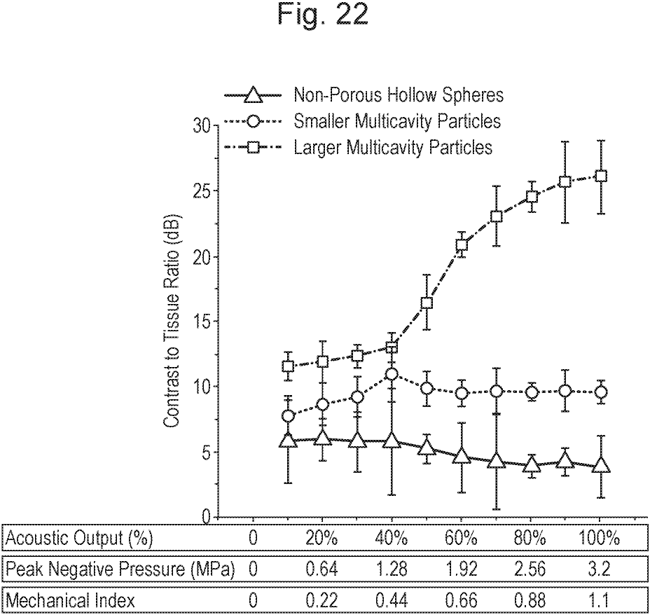

Size and the surface morphology of mcPLGA MPs were assessed using a JEOL JSM-6700 Field Emission Scanning Electron Microscope (FE-SEM; JEOL Ltd.) as described in Example 1, except in that they were coated in platinum for 3 min. The resultant images are shown in FIG. 24a. Transmission electron microscopy (TEM) was performed using a JEOL JEM 1400 operating at 120 kV. Samples for TEM imaging were prepared by adding 10 µl of aqueous dispersions on a 300-mesh carbon coated copper grids. The grids were air dried at room temperature. The resultant images are shown in FIG. 24b. Size distributions were determined by dynamic light scattering (DLS) (Malvern Nano-ZS) (FIG. 24d). To determine size changed due to HIFU, samples were exposed to 10 min of 1.1 MHz HIFU at 5% duty cycle and 3.2 MPa peak negative pressure amplitude. Fluorescence images of RhB-mcPLGA MPs were collected using a Zeiss AxioVert 200 Inverted Fluorescence Microscopy (FIG. 24c).

### Example 7: Encapsulation Efficiency and Release Profile

The quantity of Dex present and release profile as a function of time was measured by UV-absorbance in solution. A standard curve with a concentration range of 1 to 10 µM was made in PBS to correlate the mass of Dex in solution with the UV-absorbance spectral curve. The encapsulation efficiency was calculated by first measuring the remaining Dex within in the supernatant of mcPLGA MPs after solvent evaporation and subtracting it from the total amount of Dex added into the system. This difference was divided by the total amount of Dex added and multiplied by 100 to obtain the percent of Dex encapsulated.

The release of Dex in solution was performed using a sample and separation method. 5 mg of freeze-dried mcPLGA MPs was collected and dispersed in 50 ml of 0.01 M PBS (pH 7.4) buffer solution in sealed vials. This solution was maintained at 37° C. with shaking at 300 rpm. At each time point, 1 ml of the solution was taken out and centrifuged at 3000 RCF for 5 min to remove all non-dissolved solid components. The concentration of the Dex in the collected supernatant was analyzed using UV-visible spectrophotometer. The release profile was determined by the amount of Dex delivered (Mt) to the amounts of effectively encapsulated Dex (M₀), as a function of time. The experiment was performed in triplicate. The release profile is shown in FIG. 24e.

### Example 8: Acoustic Characterization

### HIFU Setup

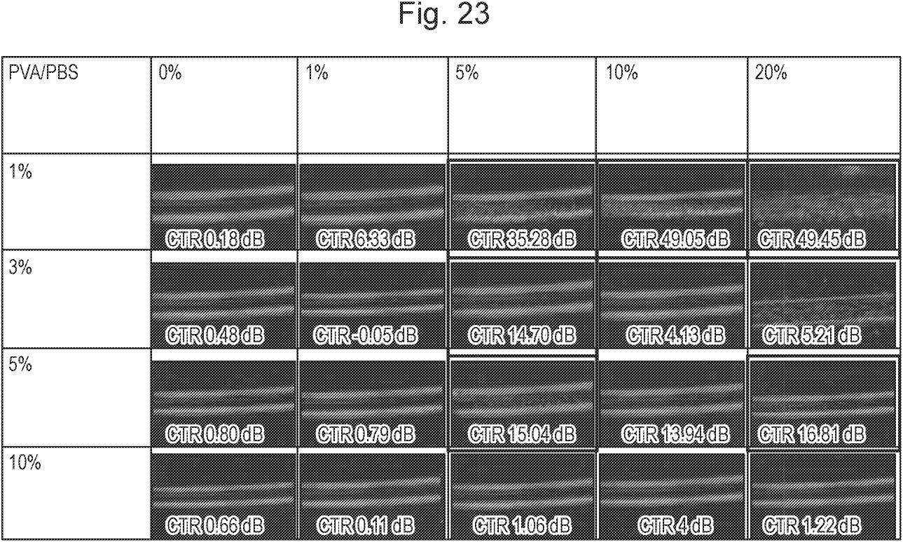

To assess the cavitation potential of the particles through a range of pressures, a custom agarose flow chamber was constructed, consisting of a 1.6 mm diameter channel in a 2 wt% agarose gel. 1 mg/ml of PLGA suspensions were constantly infused into channel at 200 µl/min. The HIFU transducer focus was set to the center of the chamber to irradiate the solution at 20 second intervals (20 cycles, 10 Hz PRF, 0.16-4.0 MPa). The cavitation response was recorded onto an oscilloscope and post processed by a power FFT to determine the power spectral density curve (FIG. 25a). For each burst, the area under the power spectral density curve was determined and compared to degassed water exposed to HIFU under the same conditions. Following the signal processing, cavitation was said to occur if the received signals were 6 dB higher than noise from the water control.

Broadband emissions indicative of inertial cavitation events were detected at pressure amplitudes above 0.9 MPa. Below 0.9 MPa peak negative pressure, bubbles from mcPLGA MPs oscillated and emitted higher harmonic emissions. Those harmonic emissions logarithmically increased with a rapid increase in the beginning and then shows as pressure amplitude increases (FIG. 25b). In contrast, the broadband emissions increased exponentially. The stable and inertial cavitation thresholds (i.e., a 50% probability for an event to occur) were measured at 0.7 MPa and 2.6 MPa, respectively. The probability for cavitation reached 100% at 1.1 MPa for stable cavitation and 3.2 MPa for inertial cavitation (FIG. 25c). Therefore, the cavitation response of mcPLGA MPs may be adjusted to achieve the desired mechanical effect by changing the input acoustic pressure.

### Diagnostic Ultrasound Setup

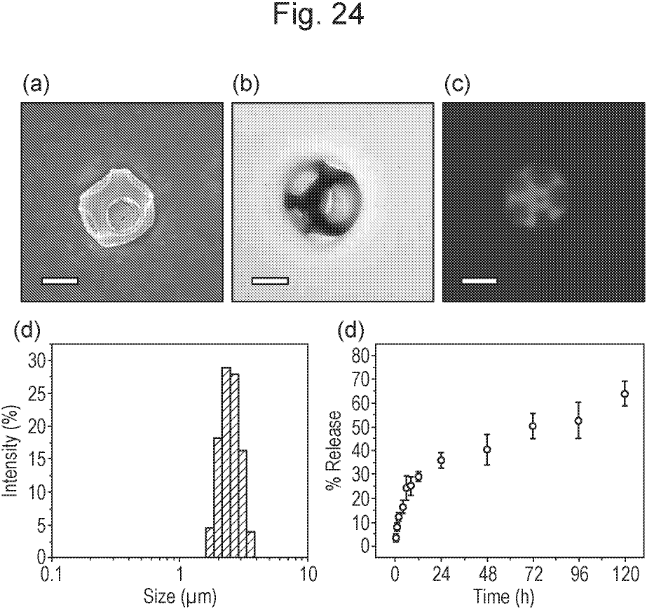

Images were obtained using an E-Cube 12-R (Alpinion Medical Systems, Seoul, South Korea) clinical ultrasound imaging system with a linear array transducer (L3-12, Alpinion Medical Systems, Seoul, South Korea) as described in Example 4. A dose of 6 mL reconstituted PLGA particles were infused into the low density polyethylene (LDPE) tube of 1.68 mm inner diameter via a syringe pump (KD Scientific KDS200, USA) at a constant rate of 1 ml/min. The LDPE tube was placed in a water bath and the probe was placed directly above it. The acoustic power was set at 60%. Images were saved in triplicate in B-Mode and save as beamformed data for each sample (FIG. 26a). As a control, deionised water was also run through the tube and images were saved. Afterwards contrast to tissue ratio (CTR) analysis was performed to quantify the ability of each PLGA particle sample to distinguish between vessel and tissue (FIG. 26b). The CTR was calculated by normalizing the mean squared acoustic power of the backscattered signal from the mcPLGA MPs suspension to the mean squared acoustic power of the backscattered signal of tissue.

Nonporous hollow spherical PLGA microparticles with the same size (hsPLGA MPs) were also compared and did not achieve substantial contrast enhancement as the mcPLGA MPs. One explanation for this discrepancy may be that while the multi-cavity particles have surface stabilised gas bubbles which can nucleate at comparatively lower input pressures, the hollow spheres have a rigid polymer shell which needs higher pressures to either induce volumetric oscillations or rupture the shell to release the gas. Hence, at the pressure range tested, the multi-cavity variant provided a higher backscattered signal from these surface stabilised gas bubbles which are missing in the hollow variant. These results indicate that mcPLGA MPs were capable of nucleating cavitation providing contrast enhancement required for clinical imaging.

### Example 9: In Vitro Foam Cell Spheroid Models

### Cell Culture and Spheroid Formation

THP-1 cells, a human monocytic cell line (ATCC, Rockville, MD) were routinely cultured in Roswell Park Memorial Institute (RPMI) 1640 Medium supplemented with 100 IU/ml of penicillin, 100 µg/ml streptomycin, 2 mmol/l L-glutamine, 10% (vol/vol) FBS and incubated in a humidified atmosphere of 5% CO₂ in an incubator at 37° C. The medium was replaced every 2-3 days by centrifuging these suspended cells at 125 g for 5 min. For differentiation of THP-1 monocytes into macrophages, THP-1 cells were seeded at a density of 7 × 10⁵ cells/ml with differentiation medium, growth medium supplemented with 50 ng/mL phorbol 12-myristate 13-acetate (PMA), for 3 days to obtain macrophages. Subsequently, for foam cells induction, macrophages were incubated with 100 µg/ml oxLDL (Low Density Lipoprotein from Human Plasma, oxidized; Invitrogen,USA) in the differentiation medium for 2 days.

Foam cells were used to produce a three-dimensional (3D) foam cell spheroid model by modification of the hanging drop method. Single-cell suspensions were generated from trypsinized monolayers. Aggregate culture of foam cells (1.25 × 106 cells/mL) were seeded into Perfecta3D® 96-well hanging drop plate (3D Biomatrix™, USA) and incubated for 4 days at 37° C. with 5% CO₂. Spheroids formed were harvested and subjected to treatment immediately afterward.

### HIFU Implantation of mcPLGA MPs Into Spheroid Studies and Ultrasound Imaging

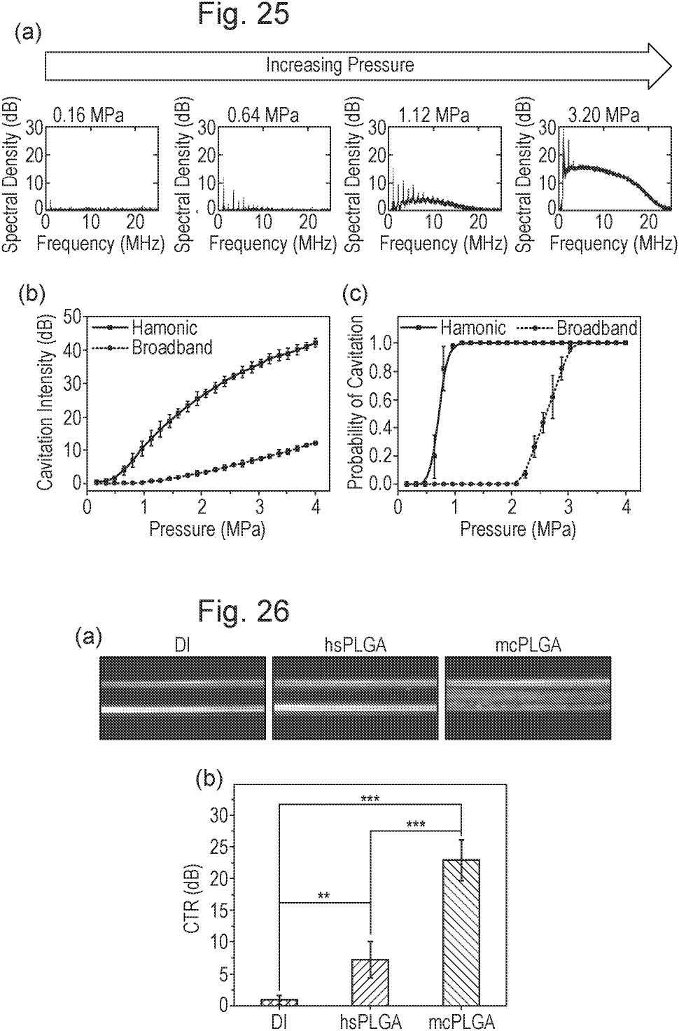

To create a spheroids embedded sample chamber for HIFU exposure, spheroids were first embedded into alginate beans. Alginate beads were generated by extruding the spheroids alginate (2%) mixture into a 10 ml of 100 mM CaCl₂. Then the beads were washed and mounted along the channel of agarose chamber mentioned above (FIG. 27a). 1 mg/ml suspension of RhB/DAPI-mcPLGA MPs were flowed through the channel using a syringe pump at a rate of 0.2 ml/min. After ultrasound exposure at 1.1 MHz center frequency, 3.2 MPa peak negative pressure, and 5% duty cycle for 5 min, the flow channel was wash with deionised water to remove the remaining mcPLGA MPs. Then the spheroids were imaged using therapeutic ultrasound setup described above (FIG. 27b) or collected for further analysis.

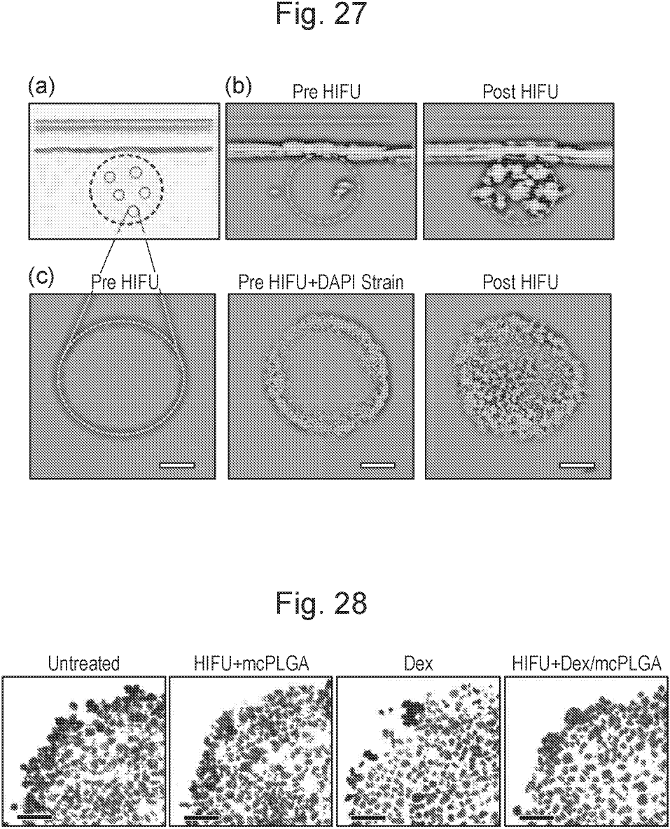

Fluorescence microscopy was used to analyze the distribution of mcPLGA MPs and the release of therapeutics (FIG. 27c). To determine the histological and physiological effects of HIFU mediated delivery of Dex loaded mcPLGA MPs to foam cell spheroids, the spheroids were sectioned (10 µm) using a cryostat. Frozen sections were stained with Oil Red O to visualize lipid under light microscopy (FIG. 28).

### Cytokine Array

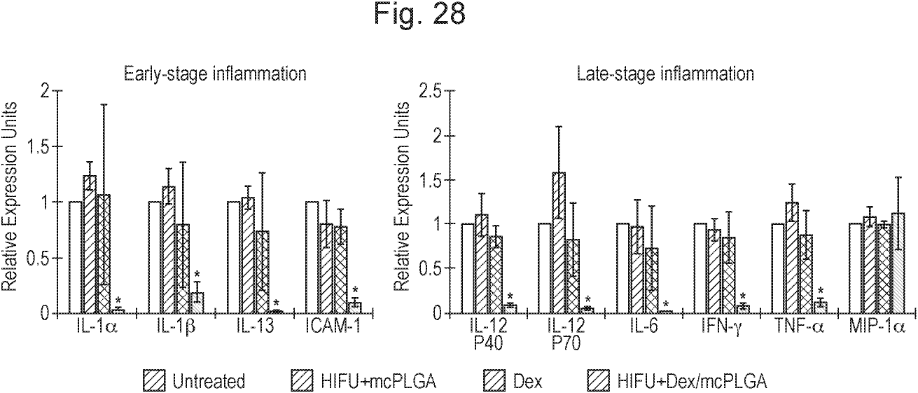

To detect secreted inflammatory cytokines, foam cell spheroid culture supernatants were collected and examined using a cytokine array (Human Cytokine Antibody Array (Membrane, 42 Targets); Abcam, USA) according to the manufacturer’s instructions. Signal intensities were quantified using the Image Quant software and normalized to the untreated samples. Evaluation of cytokine release is shown in FIG. 29.

### Statistical Analysis

All the experiments were performed in triplicates, and quantitative results were expressed as mean ± standard deviation (SD). Data were analysed by One-Way and Two-Way ANOVA with post hoc Tukey’s analysis using Origin Pro 2015 (OriginLab, USA) or student’s T test. Data presented were representative of 3 independent experiments unless otherwise specified. A p value below 0.05 was considered statistically significant.

1. A core-shell microparticle comprising a biodegradable polymer with at least two or more surface cavities for use in the treatment of vascular disease, wherein the shell further comprises one or more drugs.

2. A microparticle for use according to claim 1 wherein the treatment comprises treatment of arterial inflammation.

3. A microparticle for use according to claim 1 or claim 2 wherein the treatment comprises treatment of atherosclerosis and/or prevention of restenosis.

4. A microparticle for use according to any one of claims 1 to 3, wherein the shell comprises one or more anti-inflammatory drugs.

5. A microparticle for use according to claim 4 wherein at least one anti-inflammatory drug is a steroid, preferably dexamethasone.

6. A microparticle for use according to any one of claims 1 to 5 wherein the shell comprises an immunosuppressant drug, preferably sirolimus.

7. A microparticle for use according to any one of claims 1 to 6 wherein the biodegradable polymer is selected from an aliphatic polyester, aromatic copolyester, polyamide, poly(ester-amide), polyurethane, polyanhydride, polysaccharide, and blends or copolymers thereof.

8. A microparticle for use according to any one of claims 1 to 7 wherein the biodegradable polymer is poly(lactic-co-glycolic acid).

9. A microparticle for use according to claim 8, wherein the ratio of glycolic acid to lactic acid is from 1:4 to 4:1.

10. A microparticle for use according to any one of claims 1 to 9 wherein the average diameter of the particle is from 1 to 6 µm.

11. A microparticle for use according to any one of claims 1 to 10, wherein the core-shell microparticle is
(a) introduced into a blood vessel; and
(b) subjected to a pressure wave such that the core-shell microparticle is embedded into biological tissue.

12. A microparticle for use according to claim 11, wherein the microparticle is introduced to a diseased site within a blood vessel on the surface of a balloon, or via a catheter.

13. A microparticle for use according to claim 11 or 12, wherein the pressure wave is high intensity focused ultrasound.

14. A microparticle for use according to claim 13, wherein the pressure wave has a frequency of from 0.25 MHz to 2 MHz and a pressure of from 2 MPa to 4 MPa.

15. A microparticle for use according to any one claims 11 to 14, wherein the microparticle is subjected to ultrasound imaging after administration.

16. A microparticle for use according to claim 15, wherein the ultrasound imaging occurs at a mechanical index of 2 or less, preferably 1.2 or less.

17. A pharmaceutical composition for use in the treatment of a vascular disease as defined in any one of claims 1 to 3, wherein the pharmaceutical composition comprises a plurality of microparticles as defined in any one of claims 4 to 10 and a pharmaceutically acceptable carrier or diluent.

18. A pharmaceutical composition according to claim 17, wherein the diluent is water or an aqueous solution.

19. A core-shell microparticle comprising a biodegradable polymer with at least two or more surface cavities, wherein the shell further comprises one or more drugs, wherein the one or more drugs are selected from anti-inflammatory drugs, immunosuppressants, anti-proliferative drugs, anti-coagulants and combinations thereof.

20. A microparticle according claim 19 wherein the shell comprises a steroid, preferably dexamethasone.

21. A microparticle according to claim 19 or 20 wherein the shell comprises an immunosuppressant, preferably sirolimus.

22. A microparticle according to any one of claims 19 to 21, wherein the microparticle is defined as in any one of claims 7 to 10.

23. A pharmaceutical composition comprising a plurality of microparticles according to any one of claims 19 to 22 and a pharmaceutically acceptable carrier or diluent.

24. A pharmaceutical composition according to claim 23, wherein the diluent is water or an aqueous solution.
